SEQUENCE LISTING (93 sequences)

SEQ ID NO: 1 - protein, 832 aa, Rattus norvegicus [sequence omitted]

SEQ ID NO: 2 - protein, 832 aa, synthetic construct
  REGION 1..832: note = Description of Artificial Sequence: Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKNTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 3 - protein, 832 aa, synthetic construct
  REGION 1..832: note = Description of Artificial Sequence: Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVRHAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 4 - protein, 832 aa, synthetic construct
  REGION 1..832: note = Description of Artificial Sequence: Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVREHAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 5 - protein, 832 aa, synthetic construct
  REGION 1..832: note = Description of Artificial Sequence: Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKNTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVRHAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 6 - protein, 832 aa, synthetic construct
  REGION 1..832: note = Description of Artificial Sequence: Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKNTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVREHAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 7 - protein, 832 aa, synthetic construct
  REGION 1..832: note = Description of Artificial Sequence: Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVRHHAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 8 - protein, 832 aa, synthetic construct
  REGION 1..832: note = Description of Artificial Sequence: Synthetic ExGTq2 polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKNTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVRHHAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 9 - protein, 832 aa, synthetic construct
  REGION 1..832: note = Description of Artificial Sequence: Synthetic ExGTq polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTKKTGKRSTSAAVLEALREAHPIVEKILQYRELTKLKNTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 10 - protein, 832 aa, synthetic construct
  REGION 1..832: note = Description of Artificial Sequence: Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTKKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVRHAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 11 - protein, 832 aa, synthetic construct
  REGION 1..832: note = Description of Artificial Sequence: Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTKKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVREHAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 12 - protein, 832 aa, synthetic construct
  REGION 1..832: note = Description of Artificial Sequence: Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTKKTGKRSTSAAVLEALREAHPIVEKILQYRELTKLKNTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVRHAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 13 - protein, 832 aa, synthetic construct
  REGION 1..832: note = Description of Artificial Sequence: Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTKKTGKRSTSAAVLEALREAHPIVEKILQYRELTKLKNTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVREHAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 14 - protein, 832 aa, synthetic construct
  REGION 1..832: note = Description of Artificial Sequence: Synthetic ExGTq3 polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTKKTGKRSTSAAVLEALREAHPIVEKILQYRELTKLKNTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVRHHAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 15 - DNA, 2496 bp, synthetic construct
  misc_feature 1..2496: note = Description of Artificial Sequence: Synthetic polynucleotide
  source 1..2496: mol_type = other DNA
atgcgtgggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagaacacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaactatggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggag

SEQ ID NO: 16 - DNA, 2496 bp, synthetic construct
  misc_feature 1..2496: note = Description of Artificial Sequence: Synthetic polynucleotide
  source 1..2496: mol_type = other DNA
atgcgtgggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagaacacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaactatggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcggcatgcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggag

SEQ ID NO: 17 - DNA, 2496 bp, synthetic construct
  misc_feature 1..2496: note = Description of Artificial Sequence: Synthetic polynucleotide
  source 1..2496: mol_type = other DNA
atgcgtgggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagaacacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaactatggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcgggagcatgccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggag

SEQ ID NO: 18 - DNA, 2496 bp, synthetic construct
  misc_feature 1..2496: note = Description of Artificial Sequence: Synthetic ExGTq2 polynucleotide
  source 1..2496: mol_type = other DNA
atgcgtgggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagaacacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaactatggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcggcatcatgccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggag

SEQ ID NO: 19 - DNA, 2496 bp, synthetic construct
  misc_feature 1..2496: note = Description of Artificial Sequence: Synthetic polynucleotide
  source 1..2496: mol_type = other DNA
atgcgtgggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaactatggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcggcatgcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggag

SEQ ID NO: 20 - DNA, 2496 bp, synthetic construct
  misc_feature 1..2496: note = Description of Artificial Sequence: Synthetic polynucleotide
  source 1..2496: mol_type = other DNA
atgcgtgggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaactatggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcgggagcatgccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggag

SEQ ID NO: 21 - protein, 832 aa, synthetic construct
  REGION 1..832: note = Description of Artificial Sequence: Synthetic polypeptide
MRGMLPLFEPKGRVLLVDGHHLAYRTFHALKGLTTSRGEPVQAVYDFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 22 - DNA, 2496 bp, synthetic construct
  misc_feature 1..2496: note = Description of Artificial Sequence: Synthetic polynucleotide
  source 1..2496: mol_type = other DNA
atgcgtgggatgctgcccctctttgagcccaagggccgggtcctcctggtggacggccaccacctggcctaccgcaccttccacgccctgaagggcctcaccaccagccggggggagccggtgcaggcggtctacgatttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaactatggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggag

SEQ ID NO: 23 - protein, 832 aa, synthetic construct
  REGION 1..832: note = Description of Artificial Sequence: Synthetic polypeptide
MRGMLPLFEPKGRVLLVDGHHLAYRTFHALKGLTTSRGEPVQAVYDFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTKKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 24 - DNA, 2496 bp, synthetic construct
  misc_feature 1..2496: note = Description of Artificial Sequence: Synthetic polynucleotide
  source 1..2496: mol_type = other DNA
atgcgtgggatgctgcccctctttgagcccaagggccgggtcctcctggtggacggccaccacctggcctaccgcaccttccacgccctgaagggcctcaccaccagccggggggagccggtgcaggcggtctacgatttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacgaaaaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaactatggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggag

SEQ ID NO: 25 - protein, 832 aa, synthetic construct
  REGION 1..832: note = Description of Artificial Sequence: Synthetic polypeptide
MRGMLPLFEPKGRVLLVDGHHLAYRTFHALKGLTTSRGEPVQAVYDFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVRHHAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 26 - DNA, 2496 bp, synthetic construct
  misc_feature 1..2496: note = Description of Artificial Sequence: Synthetic polynucleotide
  source 1..2496: mol_type = other DNA
atgcgtgggatgctgcccctctttgagcccaagggccgggtcctcctggtggacggccaccacctggcctaccgcaccttccacgccctgaagggcctcaccaccagccggggggagccggtgcaggcggtctacgatttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaactatggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcggcatcatgccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggag

SEQ ID NO: 27 - protein, 832 aa, synthetic construct
  REGION 1..832: note = Description of Artificial Sequence: Synthetic polypeptide
MRGMLPLFEPKGRVLLVDGHHLAYRTFHALKGLTTSRGEPVQAVYDFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTKKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVRHHAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 28 - DNA, 2496 bp, synthetic construct
  misc_feature 1..2496: note = Description of Artificial Sequence: Synthetic polynucleotide
  source 1..2496: mol_type = other DNA
atgcgtgggatgctgcccctctttgagcccaagggccgggtcctcctggtggacggccaccacctggcctaccgcaccttccacgccctgaagggcctcaccaccagccggggggagccggtgcaggcggtctacgatttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacgaaaaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaactatggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcggcatcatgccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggag

SEQ ID NO: 29 - DNA, 2592 bp, synthetic construct
  misc_feature 1..2592: note = Description of Artificial Sequence: Synthetic polynucleotide
  source 1..2592: mol_type = other DNA
atgggcgtccagagtctgaaacgtcgccgttgcttcggcggcggtgtcactagtgggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagaacacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaactatggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcggcatcatgccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggaggcatccgaaaacctgtacttccagggccatcaccatcaccatcactga

SEQ ID NO: 30 - protein, 863 aa, synthetic construct
  REGION 1..863: note = Description of Artificial Sequence: Synthetic polypeptide
MGVQSLKRRRCFGGGVTSGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKNTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVRHHAERMAFNMPVQGTAADLMKLAMVKLFPRLEEMGARMLLQVHDELVLEAPKERAEAVARLAKEVMEGVYPLAVPLEVEVGIGEDWLSAKEASENLYFQGHHHHHH

SEQ ID NO: 31 - DNA, 2544 bp, synthetic construct
  misc_feature 1..2544: note = Description of Artificial Sequence: Synthetic polynucleotide
  source 1..2544: mol_type = other DNA
atgcgtgggatgctgcccctctttgagcccaagggccgggtcctcctggtggacggccaccacctggcctaccgcaccttccacgccctgaagggcctcaccaccagccggggggagccggtgcaggcggtctacgatttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagaacacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaactatggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcggcatcatgccgagcgcaaagccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggaggcatccgaaaacctgtacttccagggccatcaccatcaccatcactga

SEQ ID NO: 32 - protein, 847 aa, synthetic construct
  REGION 1..847: note = Description of Artificial Sequence: Synthetic polypeptide
MRGMLPLFEPKGRVLLVDGHHLAYRTFHALKGLTTSRGEPVQAVYDFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKNTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVRHHAERKAFNMPVQGTAADLMKLAMVKLFPRLEEMGARMLLQVHDELVLEAPKERAEAVARLAKEVMEGVYPLAVPLEVEVGIGEDWLSAKEASENLYFQGHHHHHH

SEQ ID NO: 33 - DNA, 34 bp, synthetic construct
  misc_feature 1..34: note = Description of Artificial Sequence: Synthetic primer
  source 1..34: mol_type = other DNA
ttcccagtcacgacgttgtaaaacgacggccagt

SEQ ID NO: 34 - protein, 6 aa, synthetic construct
  REGION 1..6: note = Description of Artificial Sequence: Synthetic 6xHis tag
HHHHHH

SEQ ID NO: 35 - protein, 15 aa, synthetic construct
  REGION 1..15: note = Description of Artificial Sequence: Synthetic peptide
ASENLYFQGHHHHHH

SEQ ID NO: 36 - DNA, 33 bp, synthetic construct
  misc_feature 1..33: note = Description of Artificial Sequence: Synthetic oligonucleotide
  source 1..33: mol_type = other DNA
ggcgtccagagtctgaaacgtcgccgttgcttc

SEQ ID NO: 37 - protein, 11 aa, synthetic construct
  REGION 1..11: note = Description of Artificial Sequence: Synthetic peptide
GVQSLKRRRCF

SEQ ID NO: 38 - DNA, 18 bp, synthetic construct
  misc_feature 1..18: note = Description of Artificial Sequence: Synthetic oligonucleotide
  source 1..18: mol_type = other DNA
ggcggcggtgtcactagt

SEQ ID NO: 39 - protein, 6 aa, synthetic construct
  REGION 1..6: note = Description of Artificial Sequence: Synthetic peptide
GGGVTS

SEQ ID NO: 40 - DNA, 66 bp, synthetic construct
  misc_feature 1..66: note = Description of Artificial Sequence: Synthetic oligonucleotide
  source 1..66: mol_type = other DNA
atgggcgtccagagtctgaaacgtcgccgttgcttcggcggcggtgtcactagtgggatgctgccc

SEQ ID NO: 41 - protein, 22 aa, synthetic construct
  REGION 1..22: note = Description of Artificial Sequence: Synthetic peptide
MGVQSLKRRRCFGGGVTSGMLP

SEQ ID NO: 42 - DNA, 141 bp, synthetic construct
  misc_feature 1..141: note = Description of Artificial Sequence: Synthetic polynucleotide
  source 1..141: mol_type = other DNA
atgcgtgggatgctgcccctctttgagcccaagggccgggtcctcctggtggacggccaccacctggcctaccgcaccttccacgccctgaagggcctcaccaccagccggggggagccggtgcaggcggtctacgatttc

SEQ ID NO: 43 - DNA, 12 bp, synthetic construct
  misc_feature 1..12: note = Description of Artificial Sequence: Synthetic oligonucleotide
  source 1..12: mol_type = other DNA
gccaaggagtaa

SEQ ID NO: 44 - DNA, 141 bp, synthetic construct; identical to SEQ ID NO: 42
  misc_feature 1..141: note = Description of Artificial Sequence: Synthetic polynucleotide
  source 1..141: mol_type = other DNA

SEQ ID NO: 45 - DNA, 57 bp, synthetic construct
  misc_feature 1..57: note = Description of Artificial Sequence: Synthetic oligonucleotide
  source 1..57: mol_type = other DNA
gccaaggaggcatccgaaaacctgtacttccagggccatcaccatcaccatcactga

SEQ ID NO: 46 - protein, 15 aa, synthetic construct
  REGION 1..15: note = Description of Artificial Sequence: Synthetic peptide
ASENLYFQGHHHHHH

SEQ ID NO: 47 - DNA, 2499 bp, Thermus aquaticus
  source 1..2499: mol_type = unassigned DNA
atgcgtgggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtaa

SEQ ID NO: 48 - protein, 832 aa, Thermus aquaticus [sequence omitted]

SEQ ID NO: 49 - DNA, 2544 bp, synthetic construct
  misc_feature 1..2544: note = Description of Artificial Sequence: Synthetic polynucleotide
  source 1..2544: mol_type = other DNA
atgcgtgggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggaggcatccgaaaacctgtacttccagggccatcaccatcaccatcactga

SEQ ID NO: 50 - protein, 847 aa, synthetic construct
  REGION 1..847: note = Description of Artificial Sequence: Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEASENLYFQGHHHHHH

SEQ ID NO: 51 - DNA, 2499 bp, synthetic construct
  misc_feature 1..2499: note = Description of Artificial Sequence: Synthetic polynucleotide
  source 1..2499: mol_type = other DNA
atgcgtgggatgctgcccctctttgagcccaagggccgggtcctcctggtggacggccaccacctggcctaccgcaccttccacgccctgaagggcctcaccaccagccggggggagccggtgcaggcggtctacgatttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaactatggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtaa

SEQ ID NO: 52 - protein, 832 aa, synthetic construct; identical to SEQ ID NO: 21
  REGION 1..832: note = Description of Artificial Sequence: Synthetic polypeptide

SEQ ID NO: 53 - DNA, 2544 bp, synthetic construct
  misc_feature 1..2544: note = Description of Artificial Sequence: Synthetic polynucleotide
  source 1..2544: mol_type = other DNA
atgcgtgggatgctgcccctctttgagcccaagggccgggtcctcctggtggacggccaccacctggcctaccgcaccttccacgccctgaagggcctcaccaccagccggggggagccggtgcaggcggtctacgatttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaactatggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggaggcatccgaaaacctgtacttccagggccatcaccatcaccatcactga

SEQ ID NO: 54 - protein, 847 aa, synthetic construct
  REGION 1..847: note = Description of Artificial Sequence: Synthetic polypeptide
MRGMLPLFEPKGRVLLVDGHHLAYRTFHALKGLTTSRGEPVQAVYDFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEASENLYFQGHHHHHH

SEQ ID NO: 55 - DNA, 2499 bp, synthetic construct
  misc_feature 1..2499: note = Description of Artificial Sequence: Synthetic polynucleotide
  source 1..2499: mol_type = other DNA
atgcgtgggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacgaaaaagaccggcaagcgctccaccagcgccgccgtcctggaggccctccgcgaggcccaccccatcgtggagaagatcctgcagtaccgggagctcaccaagctgaagaacacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaactatggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtaa

SEQ ID NO: 56 - protein, 832 aa, synthetic construct; identical to SEQ ID NO: 9
  REGION 1..832: note = Description of Artificial Sequence: Synthetic polypeptide

SEQ ID NO: 57 - DNA, 2544 bp, synthetic construct
  misc_feature 1..2544: note = Description of Artificial Sequence: Synthetic polynucleotide
  source 1..2544: mol_type = other DNA
atgcgtgggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacgaaaaagaccggcaagcgctccaccagcgccgccgtcctggaggccctccgcgaggcccaccccatcgtggagaagatcctgcagtaccgggagctcaccaagctgaagaacacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaactatggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggaggcatccgaaaacctgtacttccagggccatcaccatcaccatcactga

SEQ ID NO: 58 - protein, 847 aa, synthetic construct
  REGION 1..847: note = Description of Artificial Sequence: Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTKKTGKRSTSAAVLEALREAHPIVEKILQYRELTKLKNTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEASENLYFQGHHHHHH

SEQ ID NO: 59 - DNA, 2499 bp, synthetic construct
  misc_feature 1..2499: note = Description of Artificial Sequence: Synthetic polynucleotide
  source 1..2499: mol_type = other DNA
atgcgtgggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagaacacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaactatggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcggcatcatgccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtaa

SEQ ID NO: 60 - protein, 832 aa, synthetic construct; identical to SEQ ID NO: 8
  REGION 1..832: note = Description of Artificial Sequence: Synthetic polypeptide

SEQ ID NO: 61 - DNA, 2544 bp, synthetic construct
  misc_feature 1..2544: note = Description of Artificial Sequence: Synthetic polynucleotide
  source 1..2544: mol_type = other DNA
atgcgtgggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagaacacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaactatggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcggcatcatgccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggaggcatccgaaaacctgtacttccagggccatcaccatcaccatcactga

SEQ ID NO: 62 - protein, 847 aa, synthetic construct
  REGION 1..847: note = Description of Artificial Sequence: Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKNTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVRHHAERMAFNMPVQGTAADLMKLAMVKLFPRLEEMGARMLLQVHDELVLEAPKERAEAVARLAKEVMEGVYPLAVPLEVEVGIGEDWLSAKEASENLYFQGHHHHHH

SEQ ID NO: 63 - DNA, 2499 bp, synthetic construct
  misc_feature 1..2499: note = Description of Artificial Sequence: Synthetic polynucleotide
  source 1..2499: mol_type = other DNA
atgcgtgggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacgaaaaagaccggcaagcgctccaccagcgccgccgtcctggaggccctccgcgaggcccaccccatcgtggagaagatcctgcagtaccgggagctcaccaagctgaagaacacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaactatggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcggcatcatgccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtaa

SEQ ID NO: 64 - protein, 832 aa, synthetic construct; identical to SEQ ID NO: 14
  REGION 1..832: note = Description of Artificial Sequence: Synthetic polypeptide

SEQ ID NO: 65 - DNA, 2544 bp, synthetic construct
  misc_feature 1..2544: note = Description of Artificial Sequence: Synthetic polynucleotide
  source 1..2544: mol_type = other DNA
atgcgtgggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacgaaaaagaccggcaagcgctccaccagcgccgccgtcctggaggccctccgcgaggcccaccccatcgtggagaagatcctgcagtaccgggagctcaccaagctgaagaacacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaactatggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcggcatcatgccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggaggcatccgaaaacctgtacttccagggccatcaccatcaccatcactga

SEQ ID NO: 66 - protein, 847 aa, synthetic construct
  REGION 1..847: note = Description of Artificial Sequence: Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTKKTGKRSTSAAVLEALREAHPIVEKILQYRELTKLKNTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVRHHAERMAFNMPVQGTAADLMKLAMVKLFPRLEEMGARMLLQVHDELVLEAPKERAEAVARLAKEVMEGVYPLAVPLEVEVGIGEDWLSAKEASENLYFQGHHHHHH

SEQ ID NO: 67 - DNA, 2547 bp, synthetic construct
  misc_feature 1..2547: note = Description of Artificial Sequence: Synthetic polynucleotide
  source 1..2547: mol_type = other DNA
atgggcgtccagagtctgaaacgtcgccgttgcttcggcggcggtgtcactagtgggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagaacacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaactatggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcggcatcatgccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtaa

SEQ ID NO: 68 - protein, 848 aa, synthetic construct
  REGION 1..848: note = Description of Artificial Sequence: Synthetic polypeptide
MGVQSLKRRRCFGGGVTSGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKNTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVRHHAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 69 - DNA, 2592 bp, synthetic construct; identical to SEQ ID NO: 29
  misc_feature 1..2592: note = Description of Artificial Sequence: Synthetic polynucleotide
  source 1..2592: mol_type = other DNA

SEQ ID NO: 70 - protein, 863 aa, synthetic construct; identical to SEQ ID NO: 30
  REGION 1..863: note = Description of Artificial Sequence: Synthetic polypeptide

SEQ ID NO: 71 - DNA, 2499 bp, synthetic construct
  misc_feature 1..2499: note = Description of Artificial Sequence: Synthetic polynucleotide
  source 1..2499: mol_type = other DNA
atgcgtgggatgctgcccctctttgagcccaagggccgggtcctcctggtggacggccaccacctggcctaccgcaccttccacgccctgaagggcctcaccaccagccggggggagccggtgcaggcggtctacgatttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacgaaaaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaactatggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtaa

SEQ ID NO: 72 - protein, 832 aa, synthetic construct; identical to SEQ ID NO: 23
  REGION 1..832: note = Description of Artificial Sequence: Synthetic polypeptide

SEQ ID NO: 73 - DNA, 2544 bp, synthetic construct
  misc_feature 1..2544: note = Description of Artificial Sequence: Synthetic polynucleotide
  source 1..2544: mol_type = other DNA
atgcgtgggatgctgcccctctttgagcccaagggccgggtcctcctggtggacggccaccacctggcctaccgcaccttccacgccctgaagggcctcaccaccagccggggggagccggtgcaggcggtctacgatttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacgaaaaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaactatggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggaggcatccgaaaacctgtacttccagggccatcaccatcaccatcactga

SEQ ID NO: 74 - protein, 847 aa, synthetic construct
  REGION 1..847: note = Description of Artificial Sequence: Synthetic polypeptide
MRGMLPLFEPKGRVLLVDGHHLAYRTFHALKGLTTSRGEPVQAVYDFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTKKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEASENLYFQGHHHHHH

SEQ ID NO: 75 - DNA, 2499 bp, synthetic construct
  misc_feature 1..2499: note = Description of Artificial Sequence: Synthetic polynucleotide
  source 1..2499: mol_type = other DNA
atgcgtgggatgctgcccctctttgagcccaagggccgggtcctcctggtggacggccaccacctggcctaccgcaccttccacgccctgaagggcctcaccaccagccggggggagccggtgcaggcggtctacgatttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaactatggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcggcatcatgccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtaa

SEQ ID NO: 76 - protein, 832 aa, synthetic construct; identical to SEQ ID NO: 25
  REGION 1..832: note = Description of Artificial Sequence: Synthetic polypeptide

SEQ ID NO: 77 - DNA, 2544 bp, synthetic construct
  misc_feature 1..2544: note = Description of Artificial Sequence: Synthetic polynucleotide
  source 1..2544: mol_type = other DNA
atgcgtgggatgctgcccctctttgagcccaagggccgggtcctcctggtggacggccaccacctggcctaccgcaccttccacgccctgaagggcctcaccaccagccggggggagccggtgcaggcggtctacgatttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaactatggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcggcatcatgccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggaggcatccgaaaacctgtacttccagggccatcaccatcaccatcactga

SEQ ID NO: 78 - protein, 847 aa, synthetic construct
  REGION 1..847: note = Description of Artificial Sequence: Synthetic polypeptide
MRGMLPLFEPKGRVLLVDGHHLAYRTFHALKGLTTSRGEPVQAVYDFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVRHHAERMAFNMPVQGTAADLMKLAMVKLFPRLEEMGARMLLQVHDELVLEAPKERAEAVARLAKEVMEGVYPLAVPLEVEVGIGEDWLSAKEASENLYFQGHHHHHH

SEQ ID NO: 79 - DNA, 2499 bp, synthetic construct
  misc_feature 1..2499: note = Description of Artificial Sequence: Synthetic polynucleotide
  source 1..2499: mol_type = other DNA
atgcgtgggatgctgcccctctttgagcccaagggccgggtcctcctggtggacggccaccacctggcctaccgcaccttccacgccctgaagggcctcaccaccagccggggggagccggtgcaggcggtctacgatttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacgaaaaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaactatggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcggcatcatgccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtaa

SEQ ID NO: 80 - protein, 832 aa, synthetic construct; identical to SEQ ID NO: 27
  REGION 1..832: note = Description of Artificial Sequence: Synthetic polypeptide

SEQ ID NO: 81 - DNA, 2544 bp, synthetic construct
  misc_feature 1..2544: note = Description of Artificial Sequence: Synthetic polynucleotide
  source 1..2544: mol_type = other DNA
atgcgtgggatgctgcccctctttgagcccaagggccgggtcctcctggtggacggccaccacctggcctaccgcaccttccacgccctgaagggcctcaccaccagccggggggagccggtgcaggcggtctacgatttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacgaaaaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaactatggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcggcatcatgccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggaggcatccgaaaacctgtacttccagggccatcaccatcaccatcactga

SEQ ID NO: 82 - protein, 847 aa, synthetic construct
  REGION 1..847: note = Description of Artificial Sequence: Synthetic polypeptide
MRGMLPLFEPKGRVLLVDGHHLAYRTFHALKGLTTSRGEPVQAVYDFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTKKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVRHHAERMAFNMPVQGTAADLMKLAMVKLFPRLEEMGARMLLQVHDELVLEAPKERAEAVARLAKEVMEGVYPLAVPLEVEVGIGEDWLSAKEASENLYFQGHHHHHH

SEQ ID NO: 83 - DNA, 2499 bp, synthetic construct
  misc_feature 1..2499: note = Description of Artificial Sequence: Synthetic polynucleotide
  source 1..2499: mol_type = other DNA
atgcgtgggatgctgcccctctttgagcccaagggccgggtcctcctggtggacggccaccacctggcctaccgcaccttccacgccctgaagggcctcaccaccagccggggggagccggtgcaggcggtctacgatttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagaacacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggaaaccgccagctggatgttcggcgtcccccgggaggccgtggaccccctgatgcgccgggcggccaagaccatcaactatggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggaaaccctcttcggccgccgccgctacgtgccagacctagaggcccgggtgaagagcgtgcggcatcatgccgagcgcaaagccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtaa

SEQ ID NO: 84 - protein, 832 aa, synthetic construct
  REGION 1..832: note = Description of Artificial Sequence: Synthetic polypeptide
MRGMLPLFEPKGRVLLVDGHHLAYRTFHALKGLTTSRGEPVQAVYDFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKNTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVRHHAERKAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 85 - DNA, 2544 bp, synthetic construct; identical to SEQ ID NO: 31
  misc_feature 1..2544: note = Description of Artificial Sequence: Synthetic polynucleotide
  source 1..2544: mol_type = other DNA

SEQ ID NO: 86 - protein, 847 aa, synthetic construct; identical to SEQ ID NO: 32
  REGION 1..847: note = Description of Artificial Sequence: Synthetic polypeptide

SEQ ID NO: 87 - protein, 4 aa, synthetic construct
KRRR

SEQ ID NO: 88 - protein, 5 aa, synthetic construct
RRARR

SEQ ID NO: 89 - protein, 6 aa, synthetic construct
RRARAR

SEQ ID NO: 90 - protein, 7 aa, synthetic construct
RRAAARR

SEQ ID NO: 91 - protein, 8 aa, synthetic construct
RARARARA

SEQ ID NO: 92 - protein, 8 aa, synthetic construct
RRARAAAR

SEQ ID NO: 93 - protein, 5 aa, synthetic construct
HHHHH

(19) United States
(12) Patent Application Publication
(10) Pub. No.: US 2026/0226432 A1
(43) Pub. Date: Aug. 6, 2026

(54) ENHANCED SPEED POLYMERASES FOR SANGER SEQUENCING

(71) Applicant: THE REGENTS OF THE UNIVERSITY OF CALIFORNIA, Oakland, CA (US)

(72) Inventors: Hitomi Asahara, Oakland, CA (US); Eileen Wagner, Oakland, CA (US); Cyrus Modavi, Oakland, CA (US)

(73) Assignee: THE REGENTS OF THE UNIVERSITY OF CALIFORNIA, Oakland, CA (US)

(21) Appl. No.: 19/308,083

(22) Filed: Aug. 22, 2025

Related U.S. Application Data
(63) Continuation of application No. 17/269,222, filed on Aug. 16, 2019, now abandoned.
(60) Provisional application No. 62/719,445, filed on Aug. 17, 2018.

(51) Int. Cl. C12N 9/12; C12Q 1/6869

(52) U.S. Cl. CPC C12N 9/1252; C12Q 1/6869; C12Y 207/07007

(57) ABSTRACT

The disclosure provides compositions and methods for preparing and using modified Taq DNA polymerases. The disclosure also provides Taq DNA polymerases having improved Sanger sequencing elongation sequencing rates as compared to commercially available Sanger sequencing reagents (i.e., AmpliTaq FS™).

### PRIORITY

This application is a continuation of U.S. application Ser. No. 17/269,222, filed Feb. 17, 2021, which is a national stage entry under 35 U.S.C. § 371 of International Patent Application No. PCT/US2019/046957, filed Aug. 16, 2019, which claims priority to U.S. Provisional Patent Application No. 62/719,445, filed Aug. 17, 2018, the disclosures of which are herein incorporated by reference in their entirety for all purposes.

### SEQUENCE LISTING

The instant application contains a sequence listing that has been submitted electronically in XML format and is hereby incorporated by reference in its entirety. The XML copy, created on Apr. 15, 2026, is named 086540-1520667_007120US_SL.xml and is 230,802 bytes in size.

### FIELD OF INVENTION

The disclosure relates generally to Taq DNA polymerases for use in sequencing (e.g., Sanger sequencing). This application provides improved DNA polymerases suitable for Sanger sequencing that possess enhanced elongation speeds and the ability to sequence through secondary structures present in DNA templates. Also provided are uses for these improved DNA polymerases and methods comprising them.

### BACKGROUND

Since its introduction in 1977, Sanger sequencing has remained a dominant DNA sequencing methodology for molecular biology research and development. The DNA polymerase developed for, and commercially sold for, Sanger sequencing (AmpliTaq FS) contains proprietary modifications and requires specific formulation with other reagents to perform Sanger sequencing on Applied Biosystems (AB) sequencers.

The DNA polymerase provided by AB for Sanger sequencing (AmpliTaq FS) has a slow extension speed and has difficulties sequencing secondary structures such as GC-rich regions, hairpins, mono- and poly-nucleotide repeats. Additionally, the AmpliTaq FS DNA polymerase is only sold as part of a kit (e.g., BigDye® Terminator Cycle Sequencing Kit) needed to perform Sanger sequencing. While AB introduced specialized plastics and reductions in reaction volumes to improve Sanger sequencing reaction times, these so-called “fast thermal” cycling protocols required increased amounts of a BigDye® Terminator reagent, the most expensive reagent in the BigDye® Terminator Cycle Sequencing Kit, to compensate for low signal intensities during the sequencing reaction. Accordingly, any gains in sequencing assay performance (e.g., sequencing time or throughput) were offset by increased costs associated with the BigDye® Terminator reagent. During the last two decades, further refinement and advancement of suitable DNA polymerases to improve polymerization speeds during Sanger sequencing have been limited.

In addition to template sequencing limitations and cost, each sequencing cycle of the Sanger sequencing reaction is typically performed for 4 minutes (240 seconds), and the sequencing cycle is repeated for between 20 and 40 cycles. Thus, the time needed to perform the Sanger sequencing assay can be as short as about 80 minutes (e.g., 20 cycles at 4 minutes) to over 160 minutes (e.g., 40 cycles at 4 minutes).

Thus, there remains a need for improved DNA polymerases suitable for Sanger sequencing that possess enhanced elongation speeds, and the ability to sequence through secondary structures present in DNA templates. In some preferred aspects and embodiments, the present invention provides these and other advantages.

### BRIEF SUMMARY

In one aspect, the disclosure provides a composition comprising a Thermus aquaticus (Taq) DNA polymerase, wherein the Taq DNA polymerase comprises an F667Y substitution and at least one or more of the substitutions E742H, A743H, and S543N, and wherein the Taq DNA polymerase retains 5′ to 3′ exonuclease activity.

In some embodiments, the Taq DNA polymerase has an F667Y substitution, an E742H substitution and an A743H substitution. In some embodiments, the Taq DNA polymerase has an F667Y substitution and a S543N substitution. In some embodiments, the Taq DNA polymerase further comprises a substitution at E507K. In some embodiments, the Taq DNA polymerase has improved primer extension elongation as compared to AmpliTaq FS™. In some embodiments, the Taq DNA polymerase has improved Sanger sequencing elongation rates as compared to AmpliTaq FS™. In some embodiments, the composition further comprises a pyrophosphatase. In some embodiments, the Taq DNA polymerase has increased 5′ to 3′ exonuclease activity as compared to AmpliTaq FS™. In some embodiments, the Taq DNA polymerase has improved processivity and/or stand displacement activity as compared to AmpliTaq FS™. In some embodiments, the composition can readily incorporate a dideoxynucleotide triphosphate (ddNTP) at the 3′ end of a primer or nucleic acid molecule. In some embodiments, the composition does not discriminate between incorporation of a deoxynucleotide triphosphate (dNTP) or a dideoxynucleotide triphosphate (ddNTP) at the 3′ end of a primer or nucleic acid molecule by more than 2-fold, 3-fold, 4- fold or 5-fold (e.g., for improved results during dye-terminator sequencing). In some embodiments, the composition produces a 2-fold, 3-fold, 4-fold, 5-fold, 6-fold, 7-fold, 8-fold, or greater, reduction in sequencing cycle times.

In another aspect, the disclosure provides a polynucleotide comprising a nucleic acid sequence encoding a Taq DNA polymerase having an F667Y substitution and at least one or more of the substitutions E742H, A743H, and S543N, and wherein the Taq DNA polymerase retains 5′ to 3′ exonuclease activity.

In yet another aspect, the disclosure provides a vector comprising a polynucleotide encoding a Taq DNA polymerase having an F667Y substitution and at least one or more of the substitutions E742H, A743H, and S543N, and wherein the Taq DNA polymerase retains 5′ to 3′ exonuclease activity. In some embodiments, the vector comprises a promoter operably linked to the polynucleotide.

In one aspect, the disclosure provides a cell comprising a vector including a polynucleotide encoding a Taq DNA polymerase having an F667Y substitution and at least one or more of the substitutions E742H, A743H, and S543N, and wherein the Taq DNA polymerase retains 5′ to 3′ exonuclease activity. In some embodiments, the vector comprises a promoter operably linked to the polynucleotide.

In another aspect, the disclosure provides a method for determining a nucleic acid sequence of a nucleic acid molecule, wherein the method comprises the steps of: (1) contacting a nucleic acid molecule with a primer capable of hybridizing to the nucleic acid molecule, a ddNTP, and a Taq DNA polymerase having an F667Y substitution and at least one or more of the following substitutions E742H, A743H, and S543N, wherein the Taq DNA polymerase retains 5′ to 3′ exonuclease activity; (2) incorporating the ddNTP at the 3′ end of the primer to form an extended primer product; and (3) determining the nucleic acid sequence of the nucleic acid molecule based on the ddNTP incorporated at the 3′ end of the primer. In some embodiments, the ddNTP is ddATP, ddTTP, ddCTP, ddGTP, ddUTP, derivatives thereof, or a combination thereof. In some embodiments, the ddNTP is fluorescently labeled. In some embodiments, the ddNTP is radiolabeled. In some embodiments, the method further comprises a combination of dNTPs, where the combination is selected from two or more of dATP, dGTP, dCTP, dTTP, dUTP, and dITP. In some embodiments, the determining step includes separating the extended primer product based on molecular weight and/or capillary electrophoresis. In some embodiments, the nucleic acid sequence of the nucleic acid molecule is determined by Sanger sequencing. In some embodiments, the Sanger sequencing comprises an ddNTP incorporation step of equal to or less than 45 seconds, 30 seconds, 20 seconds, or 10 seconds. In some embodiments, the Sanger sequencing comprises an ddNTP incorporation step of equal to or less than 10 seconds. In some embodiments, the method results in a 2-fold, 3-fold, 4-fold, 5-fold, 6-fold, 7-fold, 8-fold, or greater reduction in sequencing time during the Sanger sequencing. In some embodiments, the nucleic acid sequence of the nucleic acid molecule is determined by PCR.

In one aspect, the disclosure provides a method for determining the identity of each of a series of consecutive nucleotide residues in a nucleic acid molecule, the method comprises the steps of: (a) contacting a plurality of nucleic acid molecules with a dideoxynucleotide triphosphate (ddNTP); a Taq DNA polymerase having an F667Y substitution and at least one or more of the following substitutions E742H, A743H, and S543N, and wherein the Taq DNA polymerase retains 5′ to 3′ exonuclease activity; and a primer that hybridizes to at least one of the plurality of nucleic acid molecules under conditions permitting ddNTP incorporation at the 3′ end of the primer, thereby forming a phosphodiester bond between the 3′ end of the primer and the ddNTP; (b) identifying the incorporated ddNTP, thereby identifying the consecutive nucleotide; (c) optionally, cleaving the ddNTP from the 3′ end of the primer; (d) iteratively repeating steps (a) through (c) for each of the consecutive nucleotide residues to be identified until the final consecutive nucleotide residue is to be identified; and (e) repeating steps (a) and (b) to identify the final consecutive nucleotide residue, thereby determining the identity of each of the series of consecutive nucleotide residues in the nucleic acid. In some embodiments, the ddNTP is ddATP, ddTTP, ddCTP, ddGTP, ddUTP, derivatives thereof, or a combination thereof. In some embodiments, the ddNTP comprises a plurality of ddNTP species selected from the group consisting of ddATP, ddCTP, ddGTP, ddTTP, ddUTP, derivatives thereof, and combinations thereof, and wherein each ddNTP species comprises a distinct fluorescent label. In some embodiments, the method is performed by Sanger sequencing. In some embodiments, the Sanger sequencing comprises an ddNTP incorporation step equal to or less than 30 seconds. In some embodiments, the Sanger sequencing comprises an ddNTP incorporation step equal to or less than 10 seconds. In some embodiments, the method produces an 8-fold reduction in sequencing time. In some embodiments, the contacting comprises denaturing at least one of the plurality of nucleic acid molecules, hybridizing the primer to the at least one denatured nucleic acid molecule, and extending the primer at its 3′ end by incorporation of the ddNTP. In some embodiments, step (d) is repeated for about 20 to about 40 cycles.

In one aspect, the disclosure provides a kit for nucleic acid sequencing, wherein the kit comprises a Taq DNA polymerase having an F667Y substitution and at least one or more of the following substitutions E742H, A743H, and S543N, and wherein the Taq DNA polymerase retains 5′ to 3′ exonuclease activity. In some embodiments, the kit further comprises a ddNTP. In some embodiments, the ddNTP is fluorescently labeled. In some embodiments, the ddNTP is radiolabeled. In some embodiments, the kit further comprises at least one primer. In some embodiments, the nucleic acid sequencing is Sanger sequencing. In some embodiments, the kit further comprises instructions to perform the Sanger sequencing.

### BRIEF DESCRIPTION OF THE DRAWINGS

FIG. 1 is an image of a gel showing the products of a PCR reaction for four Taq DNA polymerases prepared as disclosed herein.

FIG. 2 is an image of a gel showing the products of a PCR reaction for three Taq DNA polymerases having 5′-3′ exonuclease activity.

FIG. 3 is an image of an electropherogram showing raw sequencing data obtained via Sanger sequencing for several Taq DNA polymerases. The sequencing data was obtained using a 10-second sequencing cycle extension time.

FIG. 4 is an image of an electropherogram showing raw sequencing data obtained via Sanger sequencing for several Taq DNA polymerases. The sequencing data was obtained using a 30-second sequencing cycle extension time.

FIG. 5 is an image of an electropherogram showing raw sequencing data obtained via Sanger sequencing for several Taq DNA polymerases. The sequencing data was obtained using a 60-second sequencing cycle extension time.

FIG. 6 is an image of an electropherogram showing raw sequencing data obtained via Sanger sequencing for commercial BigDye® Sequencing reagent comprising AmpliTaq FS. The sequencing data was obtained by using sequencing extension cycles of different lengths (i.e., 10 seconds, 30 seconds, 60 seconds, 120 seconds or 240 seconds).

FIG. 7A-FIG. 7B discloses the amino acid substitutions of some Taq DNA polymerases, which includes some prior art polymerases and some embodiments of the present invention as well as some predicted structure-function correlations.

FIG. 8 is an image of an electropherogram from a Sanger sequencing speed assay comparing the Taq polymerase variants ExG2 (i.e., E742H, A743H, S543N, and F667Y mutations), ExG6 (i.e., ExGTq6 as per SEQ ID NO: 30) and TaqK (as per SEQ ID NO:32) to the commercial enzyme AmpliTaq (AmTq; AM) used in BigDye® reagent. The sequencing data was obtained by using sequencing extension times of 10, 30, and 60 seconds.

FIG. 9A-FIG. 9B is a comparison of the kinetic association rates (k_{ON}) for the Taq polymerase variants ExG2, ExG6, and TaqK and the commercial enzyme AmpliTaq (AM AmTq; AM).

FIG. 10A-FIG. 10B is a comparison of the kinetic disassociation (k_{OFF}) and surface recovery ranking (a_{OFF}) for the Taq polymerase variants ExG2, ExG6, and TaqK and the commercial enzyme AM.

FIG. 11 is a comparison of the kinetic association and disassociation rates for the Taq polymerase variants ExG2, ExG6, and TaqK and the commercial enzyme AM.

FIG. 12A-FIG. 12C is a comparison of the catalytic activity rates for the Taq polymerase variants ExG2, ExG6, and TaqK and the commercial enzyme AM.

FIG. 13 summarizes the binding kinetics and catalytic activity rates for the Taq polymerase variants ExG2, ExG6, and TaqK and the commercial enzyme AM.

### DETAILED DESCRIPTION

The disclosure relates generally to Taq DNA polymerases for use in Sanger sequencing. The Taq DNA polymerases described herein possess improved (e.g., faster) elongation rates as compared to currently available commercial Sanger sequencing DNA polymerases (i.e., AmpliTaq FS (SEQ ID NO:21)). The Taq DNA polymerases described herein can produce a reduction in sequencing cycle times needed for Sanger sequencing. In some embodiments, the Taq DNA polymerases described herein can produce a 2-fold, 3-fold, 4-fold, 5-fold, 6-fold, 7-fold, 8-fold, or greater reduction in sequencing cycle times needed for Sanger sequencing. The Taq DNA polymerases disclosed herein can be substituted for the Taq DNA polymerase provided in relevant commercially available Sanger sequencing kits (e.g., Applied Biosystems BigDye® Terminator Cycle Sequencing Kit), and do not require reformulation of the other components present in such Sanger sequencing kits. The Taq DNA polymerases provided herein produce improved sequencing output, and provide a substantial reduction in sequencing time, thus improving Sanger sequencing.

### I. Definitions

Unless defined otherwise, all technical and scientific terms used herein have the same meaning as would be commonly understood by an artisan of ordinary skill in the art to which this invention pertains.

The terms “a,” “an,” and “the” include plural referents, unless the context indicates otherwise.

The term “or” includes “and” unless the context indicates otherwise. For example, the group “A, B, or C” may include embodiments with “A and B,” “A and C,” “B and C,” and “A, B, and C” unless such a combination is not possible (e.g., alternative amino acid substitutions at the same point in a sequence).

An “amino acid” broadly refers to any monomer unit that can be incorporated into a peptide, polypeptide, or protein. As used herein, the term “amino acid” refers to an organic acid that includes a substituted or unsubstituted amino group, a substituted or unsubstituted carboxy group, and one or more side chains or groups, or analogs of any of these groups. Exemplary side chains include, e.g., thiol, seleno, sulfonyl, alkyl, aryl, acyl, keto, azido, hydroxyl, hydrazine, cyano, halo, hydrazide, alkenyl, alkynl, ether, borate, boronate, phospho, phosphono, phosphine, heterocyclic, enone, imine, aldehyde, ester, thioacid, hydroxylamine, or any combination of these groups. Other representative amino acids include, but are not limited to, amino acids comprising photoactivatable cross-linkers, metal binding amino acids, spin-labeled amino acids, fluorescent amino acids, metal-containing amino acids, amino acids with novel functional groups, amino acids that covalently or noncovalently interact with other molecules, photocaged and/or photoisomerizable amino acids, radioactive amino acids, amino acids comprising biotin or a biotin analog, glycosylated amino acids, other carbohydrate modified amino acids, amino acids comprising polyethylene glycol or polyether, heavy atom substituted amino acids, chemically cleavable and/or photocleavable amino acids, carbon-linked sugar-containing amino acids, redox-active amino acids, amino thioacid containing amino acids, and amino acids comprising one or more toxic moieties

In some preferred embodiments, the term “amino acid” includes the following twenty natural or genetically encoded alpha-amino acids: alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). In cases where “X” residues are undefined, these should be defined as “any amino acid.” The structures of these twenty natural amino acids are shown in, e.g., Stryer et al., Biochemistry, 5^{th} ed., Freeman and Company (2002). Additional amino acids, such as selenocysteine and pyrrolysine, can also be genetically coded for (Stadtman (1996) “Selenocysteine,” Annu Rev Biochem. 65:83-100 and Ibba et al. (2002) “Genetic code: introducing pyrrolysine,” Curr Biol. 12(13):R464-R466.

In some embodiments, the term “amino acid” also includes unnatural amino acids, modified amino acids (e.g., having modified side chains or backbones), and amino acid analogs. See, e.g., Zhang et al. (2004) “Selective incorporation of 5-hydroxytryptophan into proteins in mammalian cells,” Proc. Natl. Acad. Sci. U.S.A. 101(24):8882-8887, Anderson et al. (2004) “An expanded genetic code with a functional quadruplet codon” Proc. Natl. Acad. Sci. U.S.A. 101(20):7566-7571, Ikeda et al. (2003) “Synthesis of a novel histidine analogue and its efficient incorporation into a protein in vivo,” Protein Eng. Des. Sel. 16(9):699-706, Chin et al. (2003) “An Expanded Eukaryotic Genetic Code,” Science 301(5635):964-967, James et al. (2001) “Kinetic characterization of ribonuclease S mutants containing photoisomerizable phenylazophenylalanine residues,” Protein Eng. Des. Sel. 14(12):983-991, Kohrer et al. (2001) “Import of amber and ochre suppressor tRNAs into mammalian cells: A general approach to site-specific insertion of amino acid analogues into proteins,” Proc. Natl. Acad. Sci. U.S.A. 98(25):14310-14315, Bacher et al. (2001) “Selection and Characterization of Escherichia coli Variants Capable of Growth on an Otherwise Toxic Tryptophan Analogue,” J. Bacteriol. 183(18):5414-5425, Hamano-Takaku et al. (2000) “A Mutant Escherichia coli Tyrosyl-tRNA Synthetase Utilizes the Unnatural Amino Acid Azatyrosine More Efficiently than Tyrosine,” J. Biol. Chem. 275(51):40324-40328, and Budisa et al. (2001) “Proteins with {beta}-(thienopyrrolyl)alanines as alternative chromophores and pharmaceutically active amino acids,” Protein Sci. 10(7):1281-1292.

The term “mutant,” in the context of DNA polymerases of the present invention, means a polypeptide, typically recombinant, that comprises one or more amino acid substitutions relative to a corresponding, naturally-occurring or unmodified DNA polymerase.

The term “unmodified form,” in the context of a mutant polymerase, is a term used herein for purposes of identifying modifications to a known DNA polymerase. The term “unmodified form” refers to a functional DNA polymerase that has the amino acid sequence of the mutant polymerase except at one or more amino acid position(s) specified as characterizing the mutant polymerase. Thus, reference to a mutant DNA polymerase in terms of (a) its unmodified form and (b) one or more specified amino acid substitutions means that, with the exception of the specified amino acid substitution(s), the mutant polymerase otherwise has an amino acid sequence identical to the unmodified form in the specified motif. The “unmodified polymerase” may contain additional mutations to provide desired functionality, e.g., improved incorporation of dideoxyribonucleotides, ribonucleotides, ribonucleotide analogs, dye-labeled nucleotides, modulating 5′-nuclease activity, modulating 3′-nuclease (or proofreading) activity, or the like. The unmodified form of a DNA polymerase can be, for example, a wild-type and/or a naturally occurring DNA polymerase, or a DNA polymerase that has already been intentionally modified. An unmodified form of the polymerase is preferably a thermostable DNA polymerase, such as a wild-type Thermus aquaticus (Taq) DNA polymerase, as well as functional variants thereof having substantial sequence identity to a wild-type or naturally occurring thermostable polymerase.

The term “thermostable polymerase,” refers to an enzyme that is stable to heat, is heat resistant, and retains sufficient activity to effect subsequent polynucleotide extension reactions and does not become irreversibly denatured (inactivated) when subjected to the elevated temperatures for the time necessary to effect denaturation of double-stranded nucleic acids. The heating conditions necessary for nucleic acid denaturation are well known in the art and are exemplified in, e.g., U.S. Pat. Nos. 4,683,202, 4,683,195, and 4,965,188. As used herein, a thermostable polymerase is suitable for use in a temperature cycling reaction such as the polymerase chain reaction (“PCR”). Irreversible denaturation for purposes herein refers to permanent and complete loss of enzymatic activity. For a thermostable polymerase, enzymatic activity refers to the catalysis of the combination of the nucleotides in the proper manner to form polynucleotide extension products that are complementary to a template nucleic acid strand.

In the context of DNA polymerases, “correspondence” to another sequence (e.g., regions, fragments, nucleotide or amino acid positions, or the like) is based on the convention of numbering according to nucleotide or amino acid position number and then aligning the sequences in a manner that maximizes the percentage of sequence identity. Because not all positions within a given “corresponding region” need be identical, non-matching positions within a corresponding region may be regarded as “corresponding positions.” Accordingly, as used herein, referral to an “amino acid position corresponding to amino acid position [X]” of a specified DNA polymerase refers to equivalent positions, based on alignment, in other DNA polymerases and structural homologues and families. In some embodiments of the present invention, “correspondence” of amino acid positions are determined with respect to a region of the polymerase comprising one or more motifs of a sequence disclosed herein.

“Recombinant,” as used herein, refers to an amino acid sequence or a nucleotide sequence that has been intentionally modified by biotechnological methods. By the term “recombinant nucleic acid” herein is meant a nucleic acid, originally formed in vitro, in general, by the manipulation of a nucleic acid by endonucleases, in a form not normally found in nature. Thus an isolated, mutant DNA polymerase nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. A “recombinant protein” is a protein made using recombinant techniques, ie.g., through the expression of a recombinant nucleic acid as depicted above.

A nucleic acid is “operably linked” when it is placed into a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation.

The term “host cell” refers to both single-cellular prokaryote and eukaryote organisms (e.g., bacteria, yeast, and actinomycetes) and single cells from higher order plants or animals when being grown in cell culture.

The term “vector” refers to a piece of DNA, typically double-stranded, which may have inserted into it a piece of foreign DNA. The vector or may be, for example, of plasmid origin. Vectors contain “replicon” polynucleotide sequences that facilitate the autonomous replication of the vector in a host cell. Foreign DNA is defined as heterologous DNA, which is DNA not naturally found in the host cell, which, for example, replicates the vector molecule, encodes a selectable or screenable marker, or encodes a transgene. The vector is used to transport the foreign or heterologous DNA into a suitable host cell. Once in the host cell, the vector can replicate independently of or coincidental with the host chromosomal DNA, and several copies of the vector and its inserted DNA can be generated. In addition, the vector can also contain the necessary elements that permit transcription of the inserted DNA into an mRNA molecule or otherwise cause replication of the inserted DNA into multiple copies of RNA. Some expression vectors additionally contain sequence elements adjacent to the inserted DNA that increase the half-life of the expressed mRNA and/or allow translation of the mRNA into a protein molecule. Many molecules of mRNA and polypeptide encoded by the inserted DNA can thus be rapidly synthesized.

The term “nucleotide,” in addition to referring to naturally occurring ribonucleotide or deoxyribonucleotide monomers, shall herein be understood to refer to related structural variants thereof, including derivatives and analogs, that are functionally equivalent with respect to the particular context in which the nucleotide is being used (e.g., hybridization to a complementary base), unless the context indicates otherwise.

The term “nucleic acid” or “polynucleotide” refers to a polymer that can be corresponded to a ribose nucleic acid (RNA) or deoxyribose nucleic acid (DNA) polymer, or an analog thereof. This includes polymers of nucleotides such as RNA and DNA, as well as synthetic forms, modified (e.g., chemically or biochemically modified) forms thereof, and mixed polymers (e.g., including both RNA and DNA subunits). Exemplary modifications include methylation, substitution of one or more of the naturally occurring nucleotides with an analog, internucleotide modifications such as uncharged linkages (e.g., methyl phosphonates, phosphotriesters, phosphoamidates, carbamates, and the like), pendent moieties (e.g., polypeptides), intercalators (e.g., acridine, psoralen, and the like), chelators, alkylators, and modified linkages (e.g., alpha anomeric nucleic acids and the like). Also included are synthetic molecules that mimic polynucleotides in their ability to bind to a designated sequence via hydrogen bonding and other chemical interactions. Typically, the nucleotide monomers are linked via phosphodiester bonds, although synthetic forms of nucleic acids can comprise other linkages (e.g., peptide nucleic acids as described in Nielsen et al. (Science 254:1497-1500, 1991). A nucleic acid can be or can include, e.g., a chromosome or chromosomal segment, a vector (e.g., an expression vector), an expression cassette, a naked DNA or RNA polymer, the product of a polymerase chain reaction (PCR), an oligonucleotide, a probe, and a primer. A nucleic acid can be, e.g., single-stranded, double-stranded, or triple-stranded, and it is not limited to any particular length. Unless otherwise indicated, a particular nucleic acid sequence comprises or encodes complementary sequences, in addition to any sequence explicitly indicated.

The term “oligonucleotide” refers to a nucleic acid that includes at least two nucleic acid monomer units (e.g., nucleotides). An oligonucleotide typically includes from about six to about 175 nucleic acid monomer units, more typically from about eight to about 100 nucleic acid monomer units, and still more typically from about 10 to about 50 nucleic acid monomer units (e.g., about 15, about 20, about 25, about 30, about 35, about 40, or more nucleic acid monomer units). The exact size of an oligonucleotide will depend on many factors, including the ultimate function or use of the oligonucleotide. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (Meth. Enzymol. 68:90-99, 1979); the phosphodiester method of Brown et al. (Meth. Enzymol. 68:109-151, 1979); the diethylphosphoramidite method of Beaucage et al. (Tetrahedron Lett. 22:1859-1862, 1981); the triester method of Matteucci et al. (J. Am. Chem. Soc. 103:3185-3191, 1981); automated synthesis methods; the solid support method of U.S. Pat. No. 4,458,066 (Caruthers et al.), or other methods known to those skilled in the art.

The term “primer” as used herein refers to a polynucleotide capable of acting as a point of initiation of template-directed nucleic acid synthesis when placed under conditions in which polynucleotide extension is initiated (e.g., under conditions comprising the presence of requisite nucleoside triphosphates (as dictated by the template that is copied) and a polymerase in an appropriate buffer and at a suitable temperature or cycle(s) of temperatures (e.g., as in a polymerase chain reaction)). To further illustrate, primers can also be used in a variety of other oligonuceotide-mediated synthesis processes, including as initiators of de novo RNA synthesis and in vitro transcription-related processes (e.g., nucleic acid sequence-based amplification (NASBA), transcription mediated amplification (TMA), etc.). A primer is typically a single-stranded oligonucleotide (e.g., oligodeoxyribonucleotide). The appropriate length of a primer depends on the intended use of the primer but typically ranges from 6 to 40 nucleotides, more typically from 15 to 35 nucleotides. Short primer molecules generally require cooler temperatures to form sufficiently stable hybrid complexes with the template. A primer need not reflect the exact sequence of the template but must be sufficiently complementary to hybridize with a template for primer elongation to occur. In certain embodiments, the term “primer pair” means a set of primers including a 5′ sense primer (sometimes called “forward”) that hybridizes with the complement of the 5′ end of the nucleic acid sequence to be amplified and a 3′ antisense primer (sometimes called “reverse”) that hybridizes with the 3′ end of the sequence to be amplified (e.g., if the target sequence is expressed as RNA or is an RNA). A primer can be labeled, if desired, by incorporating a label detectable by spectroscopic, photochemical, biochemical, immunochemical, or chemical means. For example, useful labels include ³²P, fluorescent dyes, electron-dense reagents, enzymes (as commonly used in ELISA assays), biotin, or haptens and proteins for which antisera or monoclonal antibodies are available.

The term “conventional” or “natural” when referring to nucleic acid bases, nucleoside triphosphates, or nucleotides refers to those which occur naturally in the polynucleotide being described (i.e., for DNA these are dATP, dGTP, dCTP and dTTP). Additionally, dITP or 7-deaza-dGTP is frequently used in place of dGTP, and 7-deaza-dATP can be used in place of dATP in in vitro DNA synthesis reactions, such as sequencing. Collectively, these may be referred to as dNTPs.

The term “unconventional” or “modified” when referring to a nucleic acid base, nucleoside, or nucleotide includes modification, derivations, or analogues of conventional bases, nucleosides, or nucleotides that naturally occur in a particular polynucleotide. Certain unconventional nucleotides are modified at the 2′ position of the ribose sugar in comparison to conventional dNTPs. Thus, although for RNA the naturally occurring nucleotides are ribonucleotides (i.e., ATP, GTP, CTP, UTP, collectively rNTPs), because these nucleotides have a hydroxyl group at the 2′ position of the sugar, which, by comparison is absent in dNTPs, as used herein, ribonucleotides are unconventional nucleotides as substrates for DNA polymerases. As used herein, unconventional nucleotides include, but are not limited to, compounds used as terminators for nucleic acid sequencing. Exemplary terminator compounds include but are not limited to those compounds that have a 2′,3′ dideoxy structure and are referred to as dideoxynucleoside triphosphates. The dideoxynucleoside triphosphates ddATP, ddTTP, ddCTP and ddGTP are referred to collectively as ddNTPs. Additional examples of terminator compounds include 2′-PO4 analogs of ribonucleotides (see, e.g., U.S. Application Publication Nos. 2005/0037991 and 2005/0037398). Other unconventional nucleotides include phosphorothioate dNTPs ([[α]-S]dNTPs), 5′-[α]-borano-dNTPs, [α]-methyl-phosphonate dNTPs, and ribonucleoside triphosphates (rNTPs). Unconventional bases may be labeled with radioactive isotopes such as ³²P, ³³P, or ³⁵S; fluorescent labels; chemiluminescent labels; bioluminescent labels; hapten labels such as biotin; or enzyme labels such as streptavidin or avidin. Fluorescent labels may include dyes that are negatively charged, such as dyes of the fluorescein family, or dyes that are neutral in charge, such as dyes of the rhodamine family, or dyes that are positively charged, such as dyes of the cyanine family. Dyes of the fluorescein family include, e.g., FAM, HEX, TET, JOE, NAN and ZOE. Dyes of the rhodamine family include, e.g., Texas Red, ROX, R110, R6G, and TAMIRA. Various dyes or nucleotides labeled with FAM, HEX, TET, JOE, NAN, ZOE, ROX, R110, R6G, Texas Red, or TAMRA are marketed by Perkin-Elmer (Boston, MA), Applied Biosystems (Foster City, CA), or Invitrogen/Molecular Probes (Eugene, OR). Dyes of the cyanine family include Cy2, Cy3, Cy5, and Cy7 and are marketed by GE Healthcare UK Limited (Amersham Place, Little Chalfont, Buckinghamshire, England).

As used herein, “percentage of sequence identity” is determined by comparing two optimally aligned sequences over a comparison window, wherein the portion of the sequence in the comparison window can comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or “identity,” in the context of two or more nucleic acids or polypeptide sequences, refer to two or more sequences or subsequences that are the same. Sequences are “substantially identical” to each other if they have a specified percentage of nucleotides or amino acid residues that are the same (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% identity over a specified region), when compared and aligned for maximum correspondence over a comparison window, or designated region as measured using one of the following sequence comparison algorithms or by manual alignment and visual inspection. These definitions also refer to the complement of a test sequence. Optionally, the identity exists over a region that is at least about 50 nucleotides in length, or more typically over a region that is 100 to 500 or 1000 or more nucleotides in length.

The terms “similarity” or “percent similarity,” in the context of two or more polypeptide sequences, refer to two or more sequences or subsequences that have a specified percentage of amino acid residues that are either the same or similar as defined by a conservative amino acid substitutions (e.g., 60% similarity, optionally 65%, 70%, 75%, 80%, 85%, 86%, 87%, 88%, 89%, 90%, 91%, 92%, 93%, 94%, 95%, 96%, 97%, 98%, or 99% similarity over a specified region), when compared and aligned for maximum correspondence over a comparison window, or designated region as measured using one of the following sequence comparison algorithms or by manual alignment and visual inspection. In some embodiments, the sequences of the present invention are similar (e.g., 85%, 86%, 87%, 88%, 89%, 90%, 91%, 92%, 93%, 94%, 95%, 96%, 97%, 98%, or 99%) to a sequence set forth herein.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are commonly used, or alternative parameters can be designated. The sequence comparison algorithm then calculates the percent sequence identities or similarities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, for example, by the local homology algorithm of Smith and Waterman (Adv. Appl. Math. 2:482, 1970), by the homology alignment algorithm of Needleman and Wunsch (J. Mol. Biol. 48:443, 1970), by the search for similarity method of Pearson and Lipman (Proc. Natl. Acad. Sci. USA 85:2444, 1988), by computerized implementations of these algorithms (e.g., GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Algorithms suitable for determining percent sequence identity and sequence similarity are the BLAST and BLAST 2.0 algorithms, which are described in Altschul et al. (Nuc. Acids Res. 25:3389-402, 1977), and Altschul et al. (J. Mol. Biol. 215:403-10, 1990), respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-87, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, typically less than about 0.01, and more typically less than about 0.001.

### Polymerization

DNA sequencing often involves polymerization of a nucleotide (e.g., incorporation of a deoxynucleotide triphosphate (dNTP)) at the 3′ end of a primer that is complementary to a DNA template to be copied. Incorporation, in the context of sequencing, usually includes a denaturation step (e.g., to form single-stranded DNA molecules); an annealing/hybridization step (e.g., a primer is annealed to a complementary sequence in the single-stranded DNA molecule); and an extension step (e.g., incorporation of the dNTP at the 3′ end of the primer complementary to the single-stranded DNA molecule). Once incorporated, the process of denaturing, annealing, and extension can be repeated for additional dNTP incorporations (e.g., for between 20 and 40 cycles), and the extended primer continues to grow in length as dNTPs are incorporated.

### Sanger Sequencing

Sanger sequencing includes the above polymerization process with a notable addition: Dideoxynucleotide triphosphates (ddNTPs) are included (see U.S. Pat. No. 6,635,419). The ddNTPs lacks an 3′ OH group necessary for the formation of a 5′-3′ phosphodiester bond between the incorporated ddNTP and any additional nucleotide that attempts to incorporate. Hence, ddNTPs are often referred to as chain-terminating inhibitors of DNA polymerase. As such, the sequencing reaction is completed after an initial ddNTP incorporation. The presence of dNTPs in the Sanger sequencing reaction allows for unhindered 3′ extension of a primer, followed by termination of the extended primer product upon ddNTP incorporation (See Sanger et al., (1977) Proc. Natl. Acad. Sci. U.S.A. 74 (12): 5463-7).

### Dye-Terminator Sanger Sequencing

Dye-terminator Sanger sequencing involves labelling each species of ddNTP (e.g., ddATP, ddTTP, ddGTP, ddCTP) with a distinct signal (e.g., fluorescent dyes that emit light at different wavelengths). By labeling each species of ddNTP with a distinct signal, the Sanger sequencing reaction can be performed in a single reaction volume, as opposed to four sequencing reactions, each containing a single ddNTP species (e.g., ddATP). However, the development of fluorescently labelled ddNTPs was not well tolerated by DNA polymerases. For example, wild-type (WT) Taq DNA polymerase cannot readily incorporate labelled-ddNTPs. Accordingly, WT Taq DNA polymerase cannot be utilized for Sanger sequencing. Tabor et al. developed mutant DNA polymerases, some of which incorporated ddNTPs at least 20-fold better as compared to incorporation of the corresponding dNTPs by WT DNA polymerase (see U.S. Pat. No. 5,614,365). In some embodiments, the polymerases of the present invention incorporate ddNTPs better than WT polymerases (e.g., 2-fold, 3-fold, 4-fold, 5-fold, 6-fold, 7-fold, 8-fold, 10-fold, or more).

### Taq DNA Polymerase

The WT amino acid sequence of Taq DNA polymerase is provided as SEQ ID NO:1 (see accession number J04636). As a result of amino acid degeneracy, hundreds of different nucleotide sequences can correspond to the amino acid sequence set forth in SEQ ID NO:1. WT Taq DNA polymerase has been used in various nucleic acid amplification reactions including Polymerase Chain Reaction (PCR) (see Saiki et al., Science (1985) 1350 and Scharf, Science, (1986) 1076).

### AmpliTaq FS™

Mutant Taq DNA polymerases for PCR and Sanger sequencing are known in the art. For example, Applied Biosystems prepared a mutant Taq DNA polymerase that eliminated 5′-3′ exonuclease activity of the enzyme. The mutant Taq DNA polymerase contained a single amino acid substitution at amino acid residue 46 (i.e., G46D) (see Tabor and Richardson, Proc. Natl. Acad. Sci. USA, (1995), 92:6339-6343; Parker et al., Biotechniques (1996) 21:694-699; and Bradley, Pure & Appl. Chem., (1996) 68(10); 1907-1912) as compared to WT Taq DNA polymerase (i.e., SEQ ID NO:1).

Another single amino acid substitution in WT Taq DNA polymerase was found to be important for Sanger sequencing. Substitution of phenylalanine at amino acid residue 667 (e.g., F667Y) allowed for efficient incorporation of ddNTPs necessary for Sanger sequencing (see Tabor and Richardson, Proc. Natl. Acad. Sci. USA, (1995), 92:6339-6343). The substitution was also found to reduce background noise and maintain similar peak heights obtained in electropherograms obtained during Sanger sequencing. DNA sequencing results generated by Sanger sequencing are often provided as a plot or electropherogram, produced by an instrument (e.g., an automated DNA sequencer). The electropherogram provides a color-coded read out for each ddNTP incorporation that corresponds to the nucleic acid sequence of the nucleic acid molecule being sequenced. Accordingly, AB provided commercially available Sanger sequencing kits (e.g., BigDye® Sequencing Cycle Kit) that included a mutant Taq DNA polymerase consisting of the G46D and F667Y mutations (SEQ ID NO:21), known as Ampitaq FS™ for Sanger sequencing (see Parker et al., Biotechniques (1996) 21:694-699; Keileczawa et al., 2005 and U.S. Pat. No. 5,614,365; herein also referred to as AM).

### II. Compositions and Methods in Aspects of the Present Invention

### Improved Sanger Sequencing Elongation Rates

Surprisingly, it has now been discovered that Taq DNA polymerases possessing 5′-3′ exonuclease activity produce improved elongation rates during Sanger sequencing as compared to Taq DNA polymerases having eliminated 5′-3′ exonuclease activity (i.e., AmpliTaq FS™) Additionally, other mutations, such as E724H, A743H and S543N, introduced into WT Taq DNA polymerase were also found to result in improved elongation rates during Sanger sequencing as compared to AmpliTaq FS™. As such, in some preferred aspects, the DNA polymerases of the present invention afford these advantages.

### Compositions

In one aspect, the disclosure provides a composition comprising a Thermus aquaticus (Taq) DNA polymerase, wherein the Taq DNA polymerase comprises an F667Y substitution and at least one substitution selected from the group consisting of E507K, S543N, E742H, and A743H; and wherein the Taq DNA polymerase retains 5′ to 3′ exonuclease activity. In some preferred embodiments, the Taq DNA polymerase comprises a DNA polymerase (e.g., SEQ ID NO:1 (wild-type) or 21) that incorporates, or additionally incorporates, an F667Y substitution and at least one or more of the substitutions E507K, S543N, E742H, and A743H. In some preferred embodiments, the Taq DNA polymerase is a DNA polymerase (e.g., SEQ ID NO:1 (wild-type) or 21) that incorporates, or additionally incorporates, an F667Y substitution and other mutations as disclosed in the aspects and embodiments below.

In some embodiments, the Taq DNA polymerase as otherwise disclosed herein (e.g., a wild-type sequence with an F667K substitution) comprises at least one substitution selected from an S543N substitution, an E742H substitution, and an A743H substitution. In some embodiments, the Taq DNA polymerase comprises at least an F667K and an S543N substitution (e.g., SEQ ID NO: 2). In some embodiments, the Taq DNA polymerase comprises at least an F667K and an E742H substitution (e.g., SEQ ID NO: 3). In some embodiments, the Taq DNA polymerase comprises at least an F667K and an A743H substitution (e.g., SEQ ID NO: 4). In some embodiments, the Taq DNA polymerase comprises an F667K substitution and at least two such substitutions (e.g., S543N and E742H; E742H and A743H; or S543N and A743H) (e.g., SEQ ID NOS. 5, 7, and 6). In some embodiments, the Taq DNA polymerase comprises the substitutions F667K, S543N, E742H, and A743H [e.g., ExGTq2 (SEQ ID NO: 8)].

In some embodiments, the Taq DNA polymerase as otherwise disclosed herein (e.g., a wild-type sequence with an F667K substitution) comprises at least an E507K substitution. In some embodiments, the Taq DNA polymerase further comprises an E507K substitution. In some embodiments, the Taq DNA polymerase comprises F667Y, G46D, and E507K substitutions [e.g., AcTq (SEQ ID NO: 23)]. In some embodiments, the Taq DNA polymerase comprises F667Y, S543N, and E507K substitutions [e.g., ExGTq (SEQ ID NO: 9)]. In some embodiments, the Taq DNA polymerase comprises F667Y, S543N, E742H, A743H, and E507K substitutions [e.g., ExGTq3 (SEQ ID NO: 14)].

In some embodiments, the Taq DNA polymerase as otherwise disclosed herein (e.g., a wild-type sequence with an F667K substitution) further comprises a G46D substitution. In some embodiments, the Taq DNA polymerase comprises F667Y, E742H, A743H, and G46D substitutions [e.g., ApTq2 (“ApTaq”) (SEQ ID NO: 25)]. In some embodiments, the Taq DNA polymerase comprises F667Y, E742H, A743H, G46D, and E507K substitutions [e.g., DaTq2 (“DaTq”) (SEQ ID NO: 27)].

In some embodiments, the Taq DNA polymerase as otherwise disclosed herein (e.g., a wild-type sequence with an F667K substitution) further comprises an M747K substitution. In some embodiments, the Taq DNA polymerase comprises F667Y, S543N, E742H, A743H, G46D, and M747K substitutions [e.g., ApTq2K (“TaqK”) (SEQ ID NO: 32)].

In some embodiments, the Taq DNA polymerase as otherwise disclosed herein (e.g., a wild-type sequence with an F667K substitution) further comprises a purification tag (e.g., a histidine purification tag, such as HHHHHH (SEQ ID NO: 34)). In some embodiments, the purification tag is optionally removable, preferably without substantively affecting DNA polymerase activity. In some embodiments, the purification tag is retained, preferably without substantively affecting DNA polymerase activity. In some embodiments, the histidine purification tag comprises the sequence ASENLYFQGHHHHHH (SEQ ID NO: 35).

In some embodiments, the Taq DNA polymerase as otherwise disclosed herein (e.g., a wild-type sequence with an F667K substitution) further comprises a deletion of up to 3, 4, 5, 6, 7, 8, 9, 10, or 11 amino acids of wild-type sequence positions 1-11 (e.g., position 2; positions 2 and 3; positions 2 to 5; positions 2-11). Deletion of the amino acids indicates their from the polypeptide sequence. In some embodiments, the deleted sequence can be replaced by an alternative sequence of equal or differing length. In some embodiments, the Taq DNA polymerase as otherwise disclosed herein further comprises an R2 deletion (i.e., the residue at the 2-position).

In some embodiments of the present invention, the crystal structure of the wild-type Taq polymerase contains an unstructured N-terminal peptide chain until lysine 11. Without intending to be bound by theory, any modifications (e.g., fusion, deletion, substitution of amino acids, or substitution of a pIVc or other binding sequence) up to this point are likely not to disrupt the downstream exonuclease domain. In some embodiments, the whole Taq 5->3 exonuclease domain (approximately amino acids 1-272) can be replaced with other DNA-binding domains with no loss of enzymatic activity related to DNA polymerization.

In some embodiments, the Taq DNA polymerase as otherwise disclosed herein (e.g., a wild-type sequence with an F667K substitution) further comprises a pIVc sequence and an optional linker (e.g., at the N-terminus). In some embodiments, the pIVc sequence comprises the sequence GVQSLKRRRCF (SEQ ID NO: 37). In some embodiments, the optional linker comprises the sequence GGGVTS (SEQ ID NO: 39). In some embodiments, the N-terminal sequence comprises the sequence MGVQSLKRRRCFGGGVTSGMLP (SEQ ID NO: 41). In one embodiment, the Taq DNA polymerase further comprises S543N, E742H, and A743H substitutions as well as including a deletion at position 2, a pIVc sequence, and an optional linker (i.e., MGVQSLKRRRCFGGGVTSGMLP (SEQ ID NO: 41) at the N-terminus (e.g., as per SEQ ID NO: 30)).

Without intending to be bound by theory, the optional linker as discussed above (e.g., GGGVTS (SEQ ID NO: 39)) generally can be composed of any small or hydrophilic amino acids [e.g., peptides comprising Arg or Lys, such as KRRR (SEQ ID NO: 87), and including natural NLS (nuclear localization signal) and CPP (canonical cell-penetrating peptide) sequences]. In some embodiments, the linker is rich in Gly, Ser, or Ala. In some embodiments, the linker is one or more peptides with interleaved alanine (e.g., RRARR (SEQ ID NO: 88), RRARAR (SEQ ID NO: 89), RRAAARR (SEQ ID NO: 90), RARARARA (SEQ ID NO: 91), or RRARAAAR (SEQ ID NO: 92)). In some preferred embodiments, the linker comprises one or more small peptide sequences containing a density of lysine and residues, ideally as a block of 3 or 4, which can also be interspersed with small blocks of small peptides, fused to the N- or C-terminus of the protein.

In one aspect, the disclosure provides a composition comprising a Taq DNA polymerase, wherein the Taq DNA polymerase comprises an F667Y substitution and at least one or more of the substitutions E742H, A743H, and S543N; and wherein the Taq DNA polymerase retains 5′ to 3′ exonuclease activity. In some embodiments, the Taq DNA polymerase comprises an F667Y substitution, an E742H substitution, and an A743H substitution. In some embodiments, the Taq DNA polymerase comprises an F667Y substitution and a S543N substitution. In some embodiments, the Taq DNA polymerase comprises a DNA polymerase as otherwise disclosed herein (e.g., SEQ ID NO:1 or 21) that incorporates, or additionally incorporates, an F667Y substitution and at least one or more of the substitutions E742H, A743H, and S543N.

In some embodiments, the Taq DNA polymerase retains 5′-3′ exonuclease activity. In some embodiments, the inventive Taq DNA polymerase retains at least 50%, 55%, 60%, 65%, 70%, 75%, 80%, 85%, 90%, 95%, or more (e.g., 96%, 97%, 98%, or 99%), 5′-3′ exonuclease activity as compared to WT Taq DNA polymerase. In some embodiments, the Taq DNA polymerase possesses at least 40%, 45%, 50%, 55%, 60%, 65%, 70%, 75%, 80%, 85%, 90%, 95%, 96%, 97%, 98%, 99%, or more, 5′-3′ exonuclease activity as compared to SEQ ID NO:21 (i.e., AmpliTaq FS™)

In some embodiments, the Taq DNA polymerase does not include an amino acid substitution at residue 46 as compared to WT Taq DNA polymerase. In some embodiments, the Taq DNA polymerase does not include an amino acid substitution G46D relative to SEQ ID NO:1 (WT Taq DNA polymerase). In some embodiments, the Taq DNA polymerase does not include an N-terminal deletion relative to SEQ ID NO:1 (WT Taq DNA polymerase). In some embodiments, the Taq DNA polymerase comprises any one of SEQ ID NOS:2-14, 23, 25, 27, 30, and 32. In some embodiments, the Taq DNA polymerase comprises any one of SEQ ID NOS:2-14, 23, 25, 27, 30, 32, 48, 50, 52, 54, 56, 58, 60, 62, 64, 66, 68, 70, 72, 74, 76, 78, 80, 82, 84, and 86.

Exonuclease activity (i.e., 5′ to 3′) per mg of polymerase can be measured, for example, as described in U.S. Pat. No. 4,994,372. As set forth in U.S. Pat. No. 4,994,372, exonuclease activity was found to be detrimental to the quality of DNA sequencing reactions. Additionally, 5′ to 3′ exonuclease activity was also observed to cause DNA polymerase to idle at regions in the DNA template with secondary structures, thus the polymerase struggled to pass such regions. Thus, DNA polymerases for sequencing were developed to have preferably less than 0.1% 5′ to 3′ exonuclease activity as compared to the corresponding WT DNA polymerase.

Unexpectedly, it has now been discovered that improved sequencing throughput and reduced sequencing cycles time can be obtained by using a Taq DNA polymerase possessing 5′ to 3′ exonuclease activity. In some preferred embodiments, the Taq DNA polymerases of the present invention possess 5′ to 3′ exonuclease activity equivalent to the 5′ to 3′ exonuclease activity of the corresponding wild-type Taq DNA polymerase.

In some embodiments, the Taq DNA polymerases have improved primer extension elongation rate as compared to AmpliTaq FS™ (i.e., G46D and F667Y) under identical conditions. In some embodiments, the Taq DNA polymerases have improved Sanger sequencing elongation rates as compared to AmpliTaq FS™ (i.e., G46D and F667Y) under identical conditions. In some embodiments, the improvement in primer extension elongation rate is at least 2-fold, 3-fold, 4-fold, 5-fold, 6-fold, 7-fold, 8-fold, or more, as compared to AmpliTaq FS™ (i.e., G46D and F667Y) under identical conditions. In some embodiments, the improvement in Sanger sequencing elongation rate is at least 2-fold, 3-fold, 4-fold, 5-fold, 6-fold, 7-fold, 8-fold, or more, as compared to AmpliTaq FS™ (i.e., G46D and F667Y) under identical conditions. In some embodiments, the Taq DNA polymerases have improved primer extension elongation rates as compared to AmpliTaq FS™ under identical conditions and are selected from any one of SEQ ID NOS:2-14, 23, 25, 27, 30, and 32. In some embodiments, the Taq DNA polymerase comprises any one of SEQ ID NOS:2-14, 23, 25, 27, 30, 32, 48, 50, 52, 54, 56, 58, 60, 62, 64, 66, 68, 70, 72, 74, 76, 78, 80, 82, 84, and 86.

In some embodiments, the Taq DNA polymerase having 5′ to 3′ exonuclease activity further comprises a substitution at E507K. In some embodiments, the Taq DNA polymerase comprises any one of SEQ ID NOS:9-14, 23 and 27.

In some embodiments, the composition further comprises a pyrophosphatase (see U.S. Pat. No. 5,498,523).

In some embodiments, the Taq DNA polymerase has increased 5′ to 3′ exonuclease activity as compared to AmpliTaq FS™ (i.e., G46D and F667Y) under identical conditions. In some embodiments, the increased 5′-3′ exonuclease activity is at least 2-fold, 3-fold, 4-fold, 5-fold, or more, as compared to AmpliTaq FS™ (i.e., G46D and F667Y) under identical conditions. In some embodiments, the Taq DNA polymerase having increased 5′ to 3′ exonuclease activity as compared to AmpliTaq FS™ under identical conditions is selected from any one of SEQ ID NOS:2-14, 23, 25, 27, 30, and 32. In some embodiments, the Taq DNA polymerase comprises any one of SEQ ID NOS:2-14, 23, 25, 27, 30, 32, 48, 50, 52, 54, 56, 58, 60, 62, 64, 66, 68, 70, 72, 74, 76, 78, 80, 82, 84, and 86.

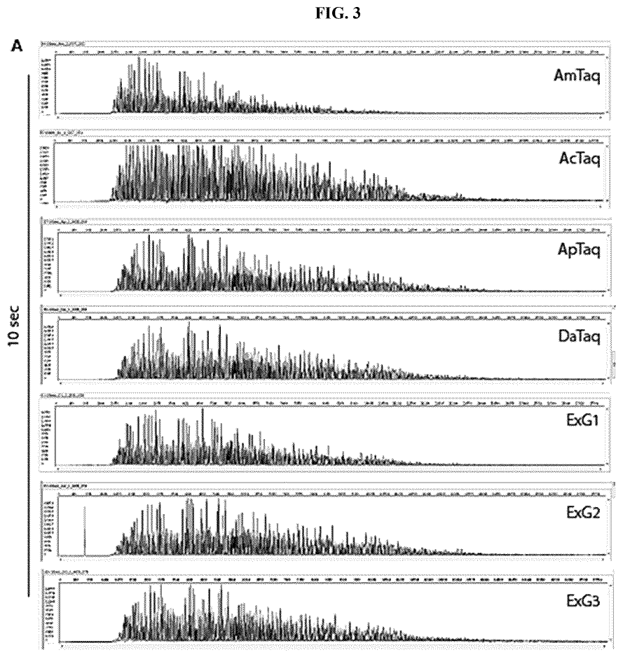

In some embodiments, the Taq DNA polymerase has improved processivity as compared to AmpliTaq FS™ under identical conditions. As used herein, “processivity” refers to the ability of a DNA polymerase to be able to continuously incorporate a plurality of nucleotides using the same primer-DNA template without dissociating from the DNA template. Processivity is known to vary among DNA polymerases. For example, T4 DNA polymerase incorporates only a few nucleotides before dissociating, while the Taq DNA polymerases of the present invention can incorporate hundreds of nucleotides before dissociating (see FIGS. 3-6). For example, in some embodiments, the Taq DNA polymerases of the present invention can sequence DNA templates having one or more secondary structures (e.g., a homopolymer of 3, 4, 5, 6, or more nucleotides, a hairpin region, or region of nucleic acids containing more than 65% GC or AT content). In some embodiments, the Taq DNA polymerases of the present invention can sequence a DNA template having a homopolymer of 3, 4, 5, 6, or more nucleotides. In some embodiments, the Taq DNA polymerases of the present invention can sequence a DNA template having a GC content of at least (or as much as) 60%, 65%, 70%, 75%, 80%, 85%, or more. In some embodiments, the Taq DNA polymerases of the present invention can sequence a DNA template having a AT content of at least (or as much as) 60%, 65%, 70%, 75%, 80%, 85%, or more. In some embodiments, the Taq DNA polymerases of the present invention can sequence a DNA template having a hairpin region. In some embodiments, the hairpin region comprises a nucleic acid sequence having a loop of 2 or more nucleotides (e.g., 2, 3, 4, 5, 6, 7, 8, or more) and a stem region of 4 or more nucleotide (e.g., 4, 5, 6, 7, 8, 9, 10, 11, 12, or more). In some embodiments, the Taq DNA polymerases of the present invention have improved processivity as compared to AmpliTaq FS™ under identical conditions and are selected from any one of SEQ ID NOS:2-14, 23, 25, 27, 30, and 32. In some embodiments, the Taq DNA polymerase comprises any one of SEQ ID NOS:2-14, 23, 25, 27, 30, 32, 48, 50, 52, 54, 56, 58, 60, 62, 64, 66, 68, 70, 72, 74, 76, 78, 80, 82, 84, and 86.

In some embodiments, the Taq DNA polymerase has improved stand displacement activity as compared to AmpliTaq FS™ under identical conditions. As used herein, “strand displacement” refers to the ability of a DNA polymerase to be able to displace downstream DNA encountered during DNA synthesis. Strand displacement is known to vary among DNA polymerases. For example, T4 and T7 DNA polymerases lack strand displacement activity, while phi29 has strong strand displacement activity. In some embodiments, the Taq DNA polymerases of the present invention have improved strand displacement activity as compared to AmpliTaq FS™ under identical conditions and are selected from any one of SEQ ID NOS:2-14, 23,25, 27, 30, and 32. In some embodiments, the Taq DNA polymerase comprises any one of SEQ ID NOS:2-14, 23, 25, 27, 30, 32, 48, 50, 52, 54, 56, 58, 60, 62, 64, 66, 68, 70, 72, 74, 76, 78, 80, 82, 84, and 86.

In some embodiments, the Taq DNA polymerases disclosed herein can incorporate a ddNTP at the 3′ end of a primer or nucleic acid molecule under Sanger sequencing reaction conditions. In some embodiments, the Taq DNA polymerases do not discriminate between incorporation of a dNTP or a ddNTP under Sanger sequencing reaction conditions by more than 2-fold, 3-fold, 4-fold or 5-fold. In some embodiments, the Taq DNA polymerases do not discriminate between incorporation of a dNTP or a ddNTP under Sanger sequencing reaction conditions by more than 5-fold.

In some embodiments, the Taq DNA polymerases provided herein are thermostable under Sanger sequencing reaction conditions.

In another aspect, the disclosure provides a polynucleotide comprising a nucleic acid sequence encoding a Taq DNA polymerase having an F667Y substitution and at least one or more of the substitutions E742H, A743H, and S543N, wherein the Taq DNA polymerase retains 5′ to 3′ exonuclease activity.

The disclosure also provides polynucleotides encoding the Taq DNA polymerases, such as SEQ ID NO: 15-20, 24, 26, 28, 29, and 31 (and, optionally, any one of SEQ ID NOS: 47, 49, 51, 53, 55, 57, 59, 61, 63, 65, 67, 69, 71, 73, 75, 77, 79, 81, 83, and 85), and cassettes and vectors including such polynucleotides. The polynucleotide may be operably linked to a promoter. Also provided are cells containing the polymerase, polynucleotides, cassettes, and/or vectors of the disclosure.

In one aspect, the disclosure provides a vector comprising a polynucleotide encoding a Taq DNA polymerase having an F667Y substitution and at least one or more of the substitutions E742H, A743H, and S543N, wherein the Taq DNA polymerase retains 5′ to 3′ exonuclease activity. In some embodiments, the vector comprises a promoter operably linked to the polynucleotide. In the polynucleotide sequences provided herein, the start codon (atg) at position 121 is underlined. Also underlined are codons that may be mutated in some embodiments of the disclosure to produce a Taq DNA polymerase of the disclosure. In some embodiments, the vector comprising a polynucleotide encoding a Taq DNA polymerase, which is selected from any one of SEQ ID NOS:15-20, 24, 26, 28, 29, and 31 (and, optionally, any one of SEQ ID NOS: 47, 49, 51, 53, 55, 57, 59, 61, 63, 65, 67, 69, 71, 73, 75, 77, 79, 81, 83, and 85). Polynucleotide sequences encoding the polymerases of the invention may be used for the recombinant production of the Taq DNA polymerases. Polynucleotide sequences encoding Taq DNA polymerases may be produced by a variety of methods. One method of producing polynucleotide sequences encoding Taq DNA polymerases is by using site-directed mutagenesis to introduce desired mutations into polynucleotides encoding the parent, wild-type Taq DNA polymerase, thereby producing a mutant (i.e., recombinant) Taq DNA polymerase.

Polynucleotides encoding the Taq DNA polymerases of the invention may be used for the recombinant expression of the Taq DNA polymerases. Generally, the recombinant expression of the Taq DNA polymerase is effected by introducing a polynucleotide encoding a Taq DNA polymerase into an expression vector adapted for use in a particular type of host cell.

Thus, another aspect of the invention is to provide vectors including a polynucleotide encoding a Taq DNA polymerase of the invention, such that the polymerase encoding polynucleotide is functionally inserted into the vector. In some embodiments, the disclosure provides a cell comprising a vector including a polynucleotide encoding a Taq DNA polymerase having an F667Y substitution and at least one or more of the substitutions E742H, A743H, and S543N, wherein the Taq DNA polymerase retains 5′ to 3′ exonuclease activity. In some embodiments, the vector comprises a promoter operably linked to the polynucleotide. In some embodiments, the vector is a plasmid. The invention also provide host cells that include the vectors of the invention. Host cells for recombinant expression may be prokaryotic or eukaryotic. Example of host cells include, but are not limited to, bacterial cells, yeast cells, cultured insect cell lines, and cultured mammalian cells lines. In some embodiments, the cell is a bacterial cell including, but not limited to, E. coli, Corynebacterium and Pseudomonas. In some embodiments, the cell is a eukaryotic cell. Examples of eukaryotic cells include, but are not limited to, S. cerevisiae, P. pastoris, and mammalian cells. In some embodiments, the mammalian cell is a human cell line (e.g., Human Embryonic Kidney (HEK) cells, human embryonic retinal cells, etc.,). A wide range of vectors, e.g., expression vectors, are well known in the art, and the expression of polymerases in recombinant cell systems is a well-established technique known to and used by those of skill in the art.

### Methods of the Present Invention

In one aspect, the disclosure provides a method for determining a nucleic acid sequence of a nucleic acid molecule, wherein the method comprises the steps of:
(1) contacting a nucleic acid molecule with a primer capable of hybridizing to the nucleic acid molecule, a ddNTP, and a Taq DNA polymerase having an F667Y substitution and at least one or more of the substitutions E742H, A743H, and S543N, wherein the Taq DNA polymerase retains 5′ to 3′ exonuclease activity;
(2) incorporating the ddNTP at the 3′ end of the primer to form an extended primer product; and
(3) determining the nucleic acid sequence of the nucleic acid molecule based on the ddNTP incorporated at the 3′ end of the extended primer product.

In some embodiments, the ddNTP is a ddNTP selected from the group consisting of ddATP, ddTTP, ddCTP, ddGTP, ddUTP, derivatives thereof, or combinations thereof. In some embodiments, the ddNTP is a combination of ddNTPs selected from two or more of ddATP, ddTTP, ddCTP, ddGTP, and ddUTP. In some embodiments, the ddNTP is labeled with a radioactive moiety (e.g., ³²P). In some embodiments, the ddNTP is fluorescently labeled. In some embodiments, the ddNTP comprises a plurality of ddNTP species, wherein each ddNTP species is fluorescently labeled with a distinct label. In some embodiments, the fluorescent label comprises a fluorescent dye. In some embodiments, each species of fluorescent label emits light at a different wavelength.

Exemplary DNA sequencing techniques include fluorescence-based sequencing methodologies (See e.g., Birren et al., Genome Analysis: Analyzing DNA, 1, Cold Spring Harbor, N.Y). Any suitable fluorophore or fluorescent dye may be used to label a ddNTP. In some embodiments, the ddNTP can include a photocleavable nucleotide. Photocleavable nucleotides include, for example, photocleavable fluorescent nucleotides and photocleavable biotinylated nucleotides. See, e.g., Li et al., PNAS, 2003, 100:414-419; Luo et al., Methods Enzymol, 2014, 549:115-131. In some embodiments, the ddNTP is fluorescently labelled with a Cy3 or Cy5 label. In some embodiments, the fluorescent label includes, but is not limited to, Alexa Fluor dyes, Fluorescein (FITC), FAM™, TET™, HEX™, JOE™, ROX™, TAMA™ and Texas Red®.

In some embodiments, the method further comprises a combination of dNTPs, where the combination of dNTPs is selected from the group consisting of dATP, dGTP, dCTP, dTTP, dUTP, and dITP, or derivatives thereof.

In some embodiments, the determining step comprises separating the extended primer product based on molecular weight and/or capillary electrophoresis. In some embodiments, the nucleic acid sequence of the nucleic acid molecule is determined by Sanger sequencing. In some embodiments, the Sanger sequencing comprises a ddNTP incorporation sequencing cycle of equal to or less than 30 seconds. In some embodiments, the Sanger sequencing comprises a ddNTP incorporation sequencing cycle of equal to or less than 10 seconds. In some embodiments, the method results in an 2-fold, 3-fold, 4-fold, 5-fold, 6-fold, 7-fold, 8-fold, or greater, reduction in ddNTP incorporation sequencing cycle time during Sanger sequencing. In some embodiments, the method results in an 8-fold reduction in sequencing time during Sanger sequencing. In some embodiments, the nucleic acid sequence of the nucleic acid molecule is determined by PCR.

In one aspect, the disclosure provides a method for determining the identity of each of a series of consecutive nucleotide residues in a nucleic acid molecule, the method comprising the steps of: (a) contacting a plurality of nucleic acid molecules with a dideoxynucleotide triphosphate (ddNTP); a Taq DNA polymerase comprising an F667Y substitution and at least one or more of the substitutions E742H, A743H, and S543N, wherein the Taq DNA polymerase retains 5′ to 3′ exonuclease activity; and a primer that hybridizes to at least one of the plurality of nucleic acid molecules under conditions permitting ddNTP incorporation at the 3′ end of the primer, thereby forming a phosphodiester bond between the 3′ end of the primer and the ddNTP; (b) identifying the incorporated ddNTP, thereby identifying the consecutive nucleotide; (c) optionally, cleaving the ddNTP from the 3′ end of the primer; (d) iteratively repeating steps (a) through (c) for each of the consecutive nucleotide residues to be identified until the final consecutive nucleotide residue is to be identified; and (e) repeating steps (a) and (b) to identify the final consecutive nucleotide residue, thereby determining the identity of each of the series of consecutive nucleotide residues in the nucleic acid. In some embodiments, the ddNTP is ddATP, ddTTP, ddCTP, ddGTP, ddUTP, or a derivative thereof. In some embodiments, the ddNTP comprises a plurality of ddNTP species selected from the group consisting of ddATP, ddCTP, ddGTP, ddTTP, and ddUTP, derivatives and combinations thereof, and wherein each ddNTP species comprises a distinct fluorescent label. In some embodiments, the method is performed by Sanger sequencing. In some embodiments, the Sanger sequencing comprises an ddNTP incorporation sequencing cycle equal to, or less than, 30 seconds. In some embodiments, the Sanger sequencing comprises an ddNTP incorporation sequencing cycle of equal to, or less than, 10 seconds. In some embodiments, the method produces an 2-fold, 3-fold, 4-fold, 5-fold, 6-fold, 7-fold, or 8-fold reduction in sequencing time. In some embodiments, the contacting comprises denaturing at least one of the plurality of nucleic acid molecules, hybridizing the primer to the at least one denatured nucleic acid molecule, and extending the primer at its 3′ end by incorporation of the ddNTP. In some embodiments, step (d) is repeated for about 20 to about 40 cycles.

In one aspect, the disclosure provides a method for purifying a Taq DNA polymerase, wherein the method comprises:
(1) contacting a polypeptide with a gel comprising cobalt, wherein the polypeptide is a Taq polymerase comprising a histidine tag;
(2) eluting the polypeptide from the gel; and
(3) optionally cleaving the polypeptide to remove the histidine tag.

In some embodiments, the histidine tag comprises the sequence HHHHH (SEQ ID NO: 93). In some embodiments, the histidine tag comprises the sequence ASENLYFQGHHHHHH (SEQ ID NO: 35). In some embodiments, the gel comprising cobalt is HisPur Cobalt Superflow Agarose gel.

### Kits

In one aspect, the disclosure provides a kit for nucleic acid sequencing, wherein the kit comprises a Taq DNA polymerase having an F667Y substitution and at least one or more of the substitutions E742H, A743H, and S543N, and wherein the Taq DNA polymerase retains 5′ to 3′ exonuclease activity. In some embodiments, the Taq DNA polymerase does not include a G46D substitution. In some embodiments, the kit further comprises a ddNTP. In some embodiments, the ddNTP is fluorescently labeled. In some embodiments, the kit further comprises at least one primer. In some embodiments, the primer is fluorescently labeled. In some embodiments, the nucleic acid sequencing is Sanger sequencing. In some embodiments, the kit further comprises instructions for performing Sanger sequencing of a nucleic acid molecule.

### EXAMPLES

### Example 1: Construction of Mutant Taq DNA Polymerases

The BigDye® Terminator Cycle Sequencing Kit (Applied Biosystems™, Catalog No. 4337450) has been the reagent of choice for Sanger sequencing for the past two decades. The kit contains a mutant Taq DNA polymerase that consists of a substitution at G46D (eliminates 5′-3′ exonuclease activity) and F667Y (allows for incorporation of ddNTPs during polymerization) called AmpliTaq FS™ (see Kieleczawa, “DNA Sequencing: Optimizing the Process and Analysis”, Vol. 1, Chapter 4 entitled “New DNA Sequencing Enzymes” (2005) ISBN-13: 9780763747824). Incorporation of a thermostable inorganic pyrophosphatase and the mutant DNA polymerase in the BigDye® Sequencing kit was found to reduce background noise and to provide better quality results. Thus, the commercial BigDye® Terminator Cycle Sequencing Kit includes both the mutant DNA polymerase (AmpliTaq FS) and an inorganic pyrophosphatase.

Here, several Taq DNA polymerases for Sanger sequencing were prepared (see Table 1). Each Taq DNA polymerase contained one or more substitutions relative to wild-type (WT) Taq DNA polymerase (SEQ ID NO:1). A list of the individual substitutions relative to WT Taq DNA polymerase and their known properties (e.g., observed during DNA polymerization) is presented in Table 1. The known effect of an F667Y mutation in WT Taq DNA polymerase is recited in the single mutation row only but is implicit to the other Taq DNA polymerases recited in Table 1. The known effects of additional mutations (e.g., E507K, E742H or A743H) are provided in Table 1.

TABLE 1

| | | |
|---|---|---|
| Single mutation | F667Y | Incorporation of ddNTPS |
| Double mutation | F667Y + E507K | Improves processivity and stabilizes |
| | | primer-template duplex structure |
| Double mutation | F667Y + E742H | Finger domain mutation to improve |
| | | polymerization speed |
| Double mutation | F667Y + A743H | Finger domain mutation to improve |
| | | polymerization speed |

### Example 2: Polymerase Expression

Plasmids containing PCR fragments encoding each of the Taq DNA polymerases were transformed into E. coli (BL21 (DE3) pROSETTA. The transformed cells were plated out onto media containing LB, ampicillin and Chloramphenicol. Individual colonies were picked from the plates and used to create an overnight starter culture in LB, ampicillin and Chloramphenicol.

After overnight incubation, 1 ml of each of the starter cultures was diluted in fresh media and incubated at 37° C. for about 3 hours. Expression of each Taq DNA polymerase was induced by adding IPTG to a final concentration of 1 mM, whereby the media was incubated for a further 3-4 hours. After which, the cells were spun in aliquots at full speed and the supernatant discarded. Cell pellets were frozen at −80° C.

The frozen cell pellets were thawed at room temperature and B-PER complete reagent was added to each cell pellet and mixed to homogeneity. The mixed samples were then incubated at room temperature for 20 minutes. After incubation, the cell mixtures were heated to 75° C. for 20 minutes to form cell lysates, with an aliquot of each cell lysate retained for SDS-PAGE confirmation of each Taq DNA polymerase. The cell lysates were centrifuged at 9,000 rpm for 20 minutes and the supernatant transferred to clean tubes for analysis.

### Example 3: Purification of His-Tagged DNA Polymerases

In order to purify the expressed Taq DNA polymerases from the supernatants of Example 2, the Taq DNA polymerases were purified by column chromatography. The following protein purification buffers were prepared: Buffer A: Equilibration buffer: 50 mM sodium phosphate, 300 mM sodium chloride, pH 7.2; and Buffer B: Elution buffer: 50 mM sodium phosphate, 300 mM sodium chloride, 90 mM imidazole, pH 7.2.

1 ml of Ni-NTA resin was placed into a clean 10 ml tube and centrifuged at 3,000 rpm, after which the supernatant was removed. Then, 6 ml of Buffer A was added to the tube, mixed, and centrifuged at 3,000 rpm. This process was repeated once more to ensure the resin was suitably equilibrated.

The lysate from Example 2 (−3 ml) was added to the resin and mixed on a shaker at room temperature for 1 hour. The resin was packed in a column and washed with 6 ml of Buffer A, collected as Flow Through. Next, the column was washed with 3 ml Buffer A, and every 1 ml was collected as Washes 1, 2 and 3. Finally, the column was washed with 6 ml of Buffer B and every 1 ml was collected.

Each fraction collected from the column was run an a SDS-PAGE gel and stained with colloidal Coomassie® blue stain. The fractions containing Taq DNA polymerase were pooled and dialyzed against 500-600 ml of dialysis buffer for several hours. The dialysis buffer was prepared as follows: 500 ml of: 50 mM TrisHCl, pH 8, 100 mM KCl, 1 mM DTT, 0.1 mM EDTA, 20% glycerol, 0.5% Tween 20, and 0.5% Nonidet P40 substitute.

The dialyzed Taq DNA polymerases were concentrated using a centrifugal filter unit with a molecular weight cutoff of 50,000 daltons. The molecular weight cutoff flow through was centrifuged at 3,000 rpm until the remaining volume was less than 250 μL, where upon the remaining volume was aliquoted into 20 μL volumes.

### Example 4: Quantitation of Purified Taq DNA Polymerases

An SDS-PAGE gel containing different dilutions of each prepared Taq DNA polymerase were assessed by diluting in 1× ThermoPol Reaction buffer (PCR protocol M0267, New England Biolabs, MA). The gel was run with 1:6 and 1:3 dilution of New England Biolab (NEB) Taq Polymerase as a control. A volume of 10 μL of dye and 10 μL diluted Taq DNA polymerase were mixed together and half of the mixture loaded onto the SDS-PAGE gel. The concentration of undiluted NEB Taq DNA polymerase was observed to be 0.055 mg/ml.

A image of the stained gel capturing areas of interest using bioinformatics software, such as ImageJ or Image Studio Lite, was performed. Areas of interest were manually selected and corresponding intensities were determined using the bioinformatics software. By accounting for dilution factors, the concentration of each purified Taq DNA polymerase was determined.

### Example 5: Taq DNA Polymerase Activity Assay

To assess the activity of each Taq DNA polymerase prepared according to the above Examples, the Taq DNA polymerases were assessed for polymerization activity. The DNA polymerases were first tested in a standard PCR reaction using various extension times. Specifically, a primer-annealed DNA template was prepared using the following DNA template and primer:
M13m 18 ssDNA template at a concentration of 1 μg/μl=0.5 M

M13 Long Primer:
(SEQ ID NO: 33)
TTCCCAGTCACGACGTTGTAAAACGACGGCCAGT

50 reactions of the annealed DNA template-primer mix in 2.5× ThermoPol buffer were prepared as a 500 μl volume as follows:
500 nM M13mpl8 ssDNA in 40 μl (20 μmol)
10 μM M13 Long primer in 40 μl (400 μmol)
10× ThermoPol Buffer (125 μl)
H₂O to 500 μl (~295 μl)

The template-primer mixture was aliquoted into five 0.2 ml tubes and underwent the following primer annealing conditions:
90° C. for 5 min;
Cooling to 70° C. at 0.1° C./s;
70° C. for 10 min;
Cooling to 4° C. at 0.1° C./s; and
Storage at −20° C.

The following polymerization activity reaction mixture were prepared:
Annealed template-primer mixture (above) 4 μl;
dNTP 0.2 μl;
H₂O 4.8 μl;
Purified mutant Taq DNA polymerase 1.0 μl

The purified mutant Taq DNA polymerases were diluted 1:100 or 1:50, including a NEB Taq DNA polymerase (control) with 1× ThermoPol Reaction buffer (20 mM TrisHCI, 10 mM (NH₄)₂SO₄, 10 mM KCl, 2 mM MgSO₄, 0.1% Triton X-100 pH 8.8) and held at 4° C. The samples were then incubated at 72° C. for 3 minutes or 5 minutes. The reactions were stopped by the addition of 1 μl 0.5 M EDTA; and the level of dNTP incorporation was quantitated using Qubit dsDNA assay. The levels of dNTP incorporation were normalized based on the level of dNTP incorporation by the NEB Taq DNA polymerase (control sample).

### Example 6: PCR Assay of Taq DNA Polymerases

The following plasmids were used in a PCR assay:

| Plasmid | Primer Pair (5′-3′) | TM | Amplicon size (bp) | GC % |
|---|---|---|---|---|
| pEL1_T4B | B68 B69 | 64 | 1458 | 36 |
| pEL1_T4B | DQ60 DQ48 | 60 | 238 | 55 |
| pEL1_T4B | D26 BT31 | 55 | 2003 | 43 |
| pGEM-3Zfp | AD78 AW39 | 55 | 2968 | 50 |
| pGEM-3Zfp | BT31 AW39 | 55 | 2523 | 49 |
| pGEM-3Zfp | M13F pGEMR | 50 | 1018 | NA |
| pGEM-3Zfp | M13F AW39 | 50 | 535 | 52 |
| pGEM-3Zfp | M13F Ml3R | 50 | 155 | 48 |

The following reaction mixture was prepared for each PCR assay:

| | |
|---|---|
| 10x ThermoPol buffer | 2.5 μl |
| 10 mM dNTP | 0.5 μl |
| 10 uM Forward (F) primer | 0.5 μl |
| 10 uM Reverse (R) primer | 0.5 μl |
| Plasmid (5 ng/ul) | 1 μl |
| diluted polymerase | 1 μl |
| H₂O | 19 μl |

Adjustment of reaction volume to 25 μl

Each reaction mixture underwent the following PCR conditions:
1 cycle at 95° C. for 1 min; followed by 35 cycles of the following steps:
95° C. for 15 seconds
Annealing step for between 10-60 seconds
Extension step at 68° C. for between 10-60 seconds.

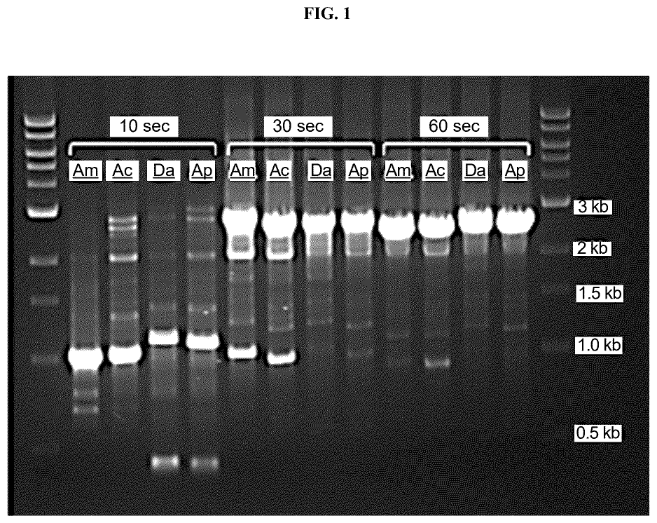

The PCR reactions were stopped and run on a 1.2% agarose gel to evaluate amplicon size and quantity (see FIG. 1).

Referring to FIG. 1, the image shows the results of the PCR assay described above, for four different Taq DNA polymerases prepared as disclosed herein. Five units of each polymerase were used to amplify a 2.5 kb fragment from pGEM-3Zfp using 10-, 30-, or 60-second extension times. In FIG. 1, “Am” refers to “AmTaq” (AmpliTaq FS (i.e., G46D and F667Y mutations); “Ac” refers to “AcTaq” (i.e., E507K+F667Y+G46D mutation); “Da” refers to “DaTaq” (i.e., E507K+F667Y+G46D+, E742H and A743H mutations); and Ap refers to “ApTaq” (i.e., F667Y+G46D+E742H and A743H mutations). As is evident from FIG. 1, Ap and Da outperformed Am and Ac as evidenced by truncated PCR products formed by the latter polymerases, for example, in the 30-second extension time.

A similar experiment was performed using three different Taq DNA polymerases in which the 5′-3′ exonuclease had been restored. To prepare a polymerase having 5′-3′ exonuclease activity (unlike AmpliTaq FS), the G46D substitution was reverted to wild-type (i.e., G46). Each of the 5′-3′ exonuclease activity Taq DNA polymerases were prepared essentially as described herein.

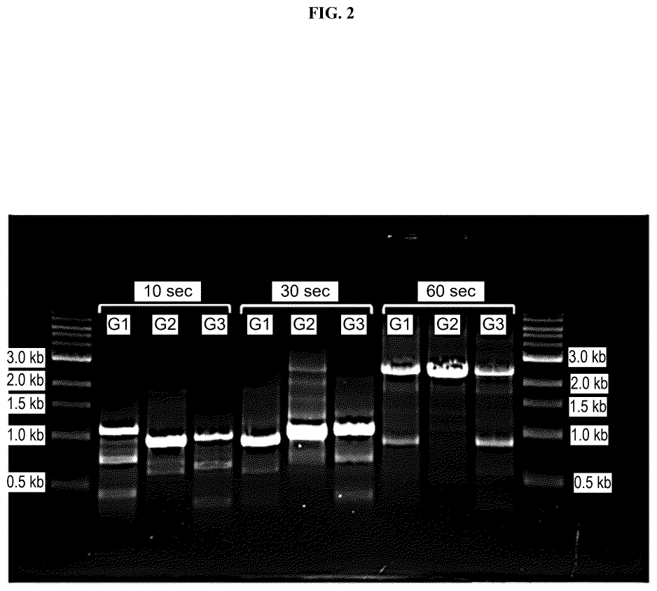

Referring to FIG. 2, the image shows the results of a PCR assay for the three different Taq DNA polymerases having 5′-3′ exonuclease activity. Five units of each prepared Taq DNA polymerase were used to amplify a 2.5 kb fragment from pGEM-3Zfp using 10-, 30-, or 60-second extension times. In FIG. 2, “G1” refers to “ExG1” (i.e., E507K, S543N, and F667Y mutations); “G2” refers to “ExG2” (i.e., E742H, A743H, S543N, and F667Y mutations); and G3 refers to “ExG3” (i.e., E507K, E742H, A743H, S543N, and F667Y mutations). As is evident from FIG. 2, G2 outperformed G1 and G3 as evidenced by truncated PCR products formed by the latter polymerases, for example, in the 60-second extension time.

### Example 7: Sanger Sequencing of Taq DNA Polymerases

The commercially available Sanger polymerase provided Sequencing kit includes a polymerase (AmpliTaq FS™). This polymerase was treated with proteinase K to destroy polymerase activity, prior to adding an aliquot of each of the Taq DNA polymerases disclosed herein for testing and evaluation. The Sanger sequencing assay for each of the Taq DNA polymerase were performed as follows:

### Proteinase K Treatment of BigDye® Reagent

3 μl of Proteinase K (ThermoFisher Scientific, 20 mg/ml) was added to 67 μl of BigDye® Kit Reagent and incubated for 20 minutes at 37° C. The proteinase K was then heat inactivated at 95° C. for 10 minutes before standard BigDye® sequencing reaction mixtures were prepared.

### Standard BigDye® Sequencing Reaction Mixture

The Proteinase K treated BigDye® reagent was diluted 1:12 with ABI 5x Sequencing buffer (i.e., 70 μl proteinase K BigDye® treated reagent, 167 μl of ABI 5× Buffer and 167 μl H₂O).

dGTP BigDye® Sequencing Reaction Mixture

All Taq DNA polymerases (control and Taq DNA polymerases of the present invention) were diluted to 1 unit/μl with 1× ThermoPol Buffer and 1 unit of the diluted Taq DNA polymerase were used to sequence various plasmids described in Example 6 using a standard Sanger sequencing protocol or dGTP BigDye® Sequencing protocol (outlined below).

### Standard Sanger Sequencing BigDye® Sequencing Cycle Protocol

5 μl of plasmid (e.g., pGEM) was mixed with 4 μl of the 1:12 diluted proteinase K treated BigDye® reagent and 1 μl of Taq DNA polymerase. The reaction mixture was then placed under the following PCR conditions:
1 cycle at 96° C. for 1 minute;
followed by 25 cycles at: 96° C. for 10 seconds;
   50° C. for 5 seconds;
   60° C. for a variable time period; and held at 12° C.
dGTP BigDye@Sequencing Cycle Protocol

4 μl of plasmid (e.g., pGEM) was mixed with 2 μl betaine and heat denatured at 98° C. for 5 minutes followed by cooling on ice for 5 minutes. After which, 4 μl of a 1:8 dilution of the dGTP BigDye® reagent was added and placed under the following PCR conditions:
35 cycles at 98° C. for 10 seconds;
50° C. for 5 seconds;
60° C. for a variable time period; and held at 12° C.

The samples were run on an Applied Biosystems Sequencer (ABI 3730XL), and sequence data was analyzed using ABI Data Analysis software.

Sequencing cycle extension times of 10, 30 and 60 seconds were tested, using pGEM-3Zfp as the DNA template and compared the results to the standard Sanger sequencing reaction using BigDye® Terminator reagent (i.e., AmpliTaq FS) with a 240-second extension time.

The results of the Sanger sequencing assay are provided in FIGS. 3-6.

In FIG. 3, raw sequencing data is provided for each of the Taq DNA polymerases of the present invention based on a 10-second extension time. All of the prepared Taq DNA polymerases produced longer sequencing reads than AmpliTaq FS (i.e., AmTaq) under the 10-second extension period.

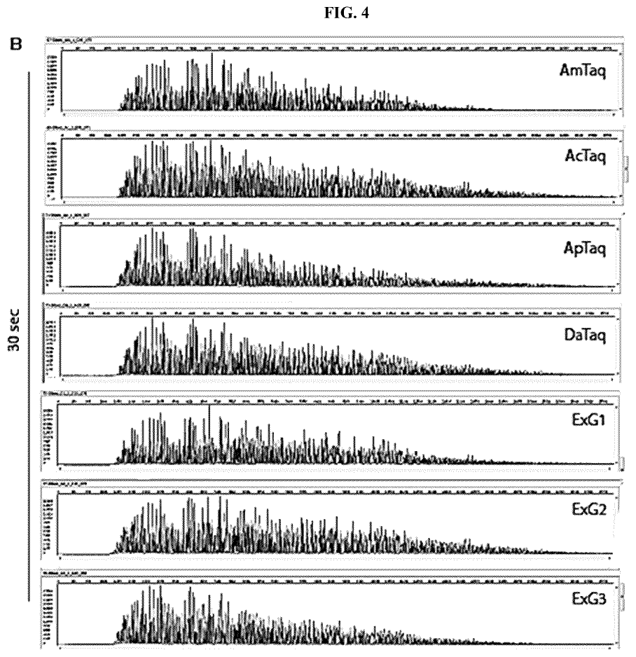

In FIG. 4, raw sequencing data is provided for each of the Taq DNA polymerases of the present invention based on a 30-second extension time. The prepared Taq DNA polymerases AcTaq, ApTaq, DaTaq and ExG2 produced longer sequencing reads than AmpliTaq FS (i.e., AmTaq) under the 30-second extension period.

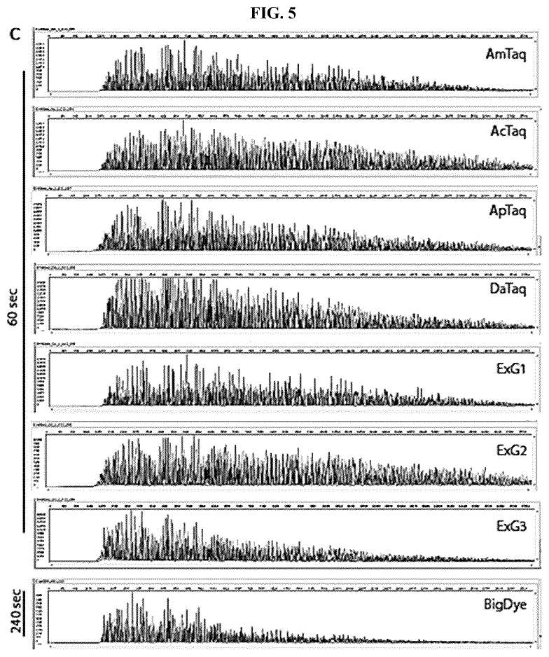

In FIG. 5, raw sequencing data is provided for each of the Taq DNA polymerases of the present invention based on a 60-second extension time. The prepared Taq DNA polymerases AcTaq, ApTaq, DaTaq ExG1 and ExG2 produced longer sequencing reads than AmpliTaq FS (i.e., AmTaq) under the 60-second extension period. In FIG. 5, the commercial BigDye® Sequencing reagent containing AmpliTaq FS not treated with proteinase K, is shown as a control, and included with the standard “240-second” extension time recommended for the BigDye® Terminator Sequencing Cycle protocol.

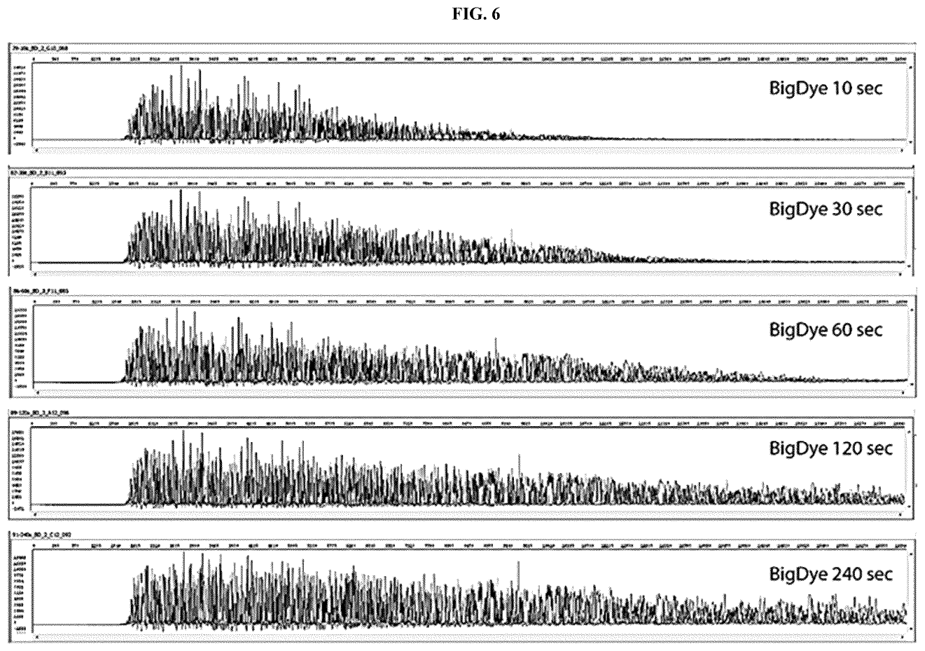

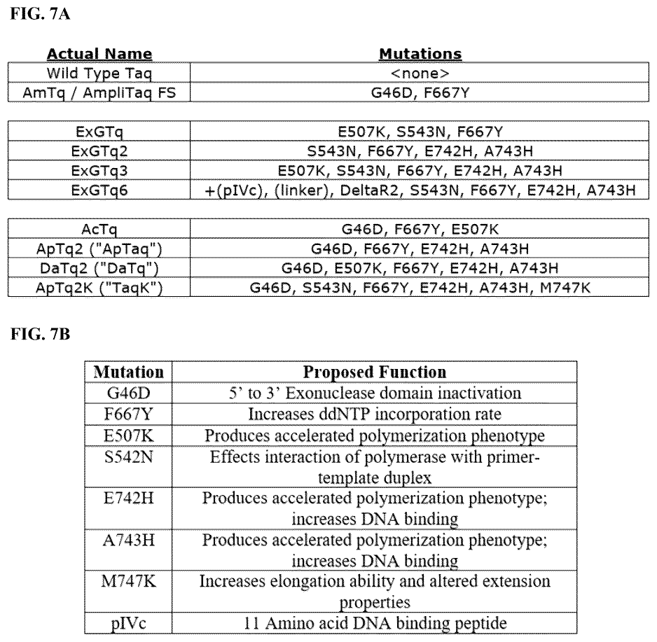

In FIG. 6, raw sequencing data is provided for the commercial BigDye® Sequencing reagent comprising AmpliTaq FS. Here, sequencing data was obtained by using sequencing extension cycles of different lengths (i.e., 10 seconds, 30 seconds, 60 seconds, 120 seconds, or 240 seconds). Full length product was only obtained for the commercial BigDye® Sequencing reagent at 120 seconds.

Several of the Taq DNA polymerases of the present invention (e.g., AcTaq, ApTaq, DaTaq and Exg2), produced full length sequencing reads within the 30-second extension period. In contrast, the BigDye® reagents used as a control Taq DNA polymerase required the 240-second extension period to obtain full length sequencing reads. Thus, the Taq DNA polymerases of the present invention can be used for Sanger sequencing and result in a reduction in sequencing time as compared to the currently available commercial reagent (AmpliTaq FS) used in Sanger sequencing. In some embodiments, the Taq DNA polymerases of the present invention can result in a 2-fold, 3-fold, 4-fold, 5-fold, 6-fold, 7-fold, 8-fold, or greater reduction in Sanger sequencing cycle times.

### Example 8: Alternative Purification Method for DNA Polymerases

A second, alternative column chromatography method was used to purify the expressed Taq DNA polymerases from the supernatants of Example 2. The following protein purification buffers were prepared:
Buffer A: Binding buffer: 20 mM sodium phosphate, 300 mM sodium chloride, pH 7.2;
Buffer B: Wash buffer: 20 mM sodium phosphate, 300 mM sodium chloride, 90 mM imidazole, pH 7.2; and
Buffer C: Elution buffer: 20 mM sodium phosphate, 300 mM sodium chloride, 300 mM imidazole, pH 7.2.

The typical yield of a cell pellet from approximately 250 ml cell culture is 350~500 mg. In a representative he cells were lysed with 2~3 ml of BugBuster Master mix, which typically results in 3-4 ml of cleared lysate.

1 to 2 ml of HisPur™ Cobalt Superflow Agarose resin was placed into a clean 15 ml tube tube depending on the volume of cleared lysate (e.g., 3 to 4 ml) and centrifuged at 3,000 rpm, after which the supernatant was carefully removed. Then 6 ml of Buffer A was added to the tube, mixed, and centrifuged at 3,000 rpm. This process was repeated once more to ensure the resin was suitably equilibrated.

The lysate from Example 2 (~3 ml) was added to the resin and mixed on a shaker at room temperature for 1 hour. The resin was packed in a column and washed with 6 ml of Buffer A, collected as Flow Through. Next, the column was washed with 3×1 ml Buffer A, and every 1 ml fraction was collected. The column was then washed with 3×1 ml Buffer B, and every 1 ml fraction was collected. Finally, the column was washed with 6×1 ml of Buffer C, and every 1 ml fraction was collected.

Each fraction collected from the column was run an a SDS-PAGE gel and stained with Imperial Protein stain. The fractions containing Taq DNA polymerase were pooled and dialyzed against ca. 1 L of dialysis buffer overnight. The dialysis buffer was prepared as follows: 500 ml of: 50 mM TrisHCl, pH 8, 100 mM KCl, 1 mM DTT, 0.1 mM EDTA, 20% glycerol, 0.5% Tween 20, and 0.5% Nonidet P40 substitute.

The dialyzed Taq DNA polymerases were concentrated using an Amicon Ultra4 filter unit with a molecular weight cutoff of 50,000 daltons. The molecular weight cutoff flow through was centrifuged at 3,000 rpm until the remaining volume was less than 300 μL.

### Example 9: Sanger Sequencing of Taq DNA Polymerases II

A Sanger sequencing assay was conducted for four Taq polymerases: AM, ExG2, ExG6, and TaqK. The procedure according to Example 7 was used upon solutions of the four Taq polymerases to be sequenced. Sequencing cycle extension times of 10, 30 and 60 seconds were tested, using pGEM-3Zfp as the DNA template.

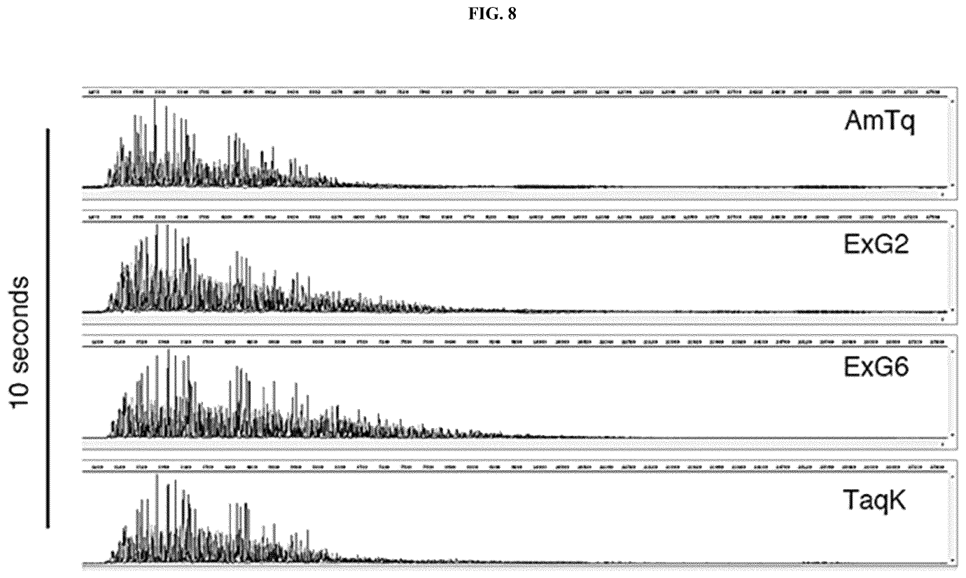

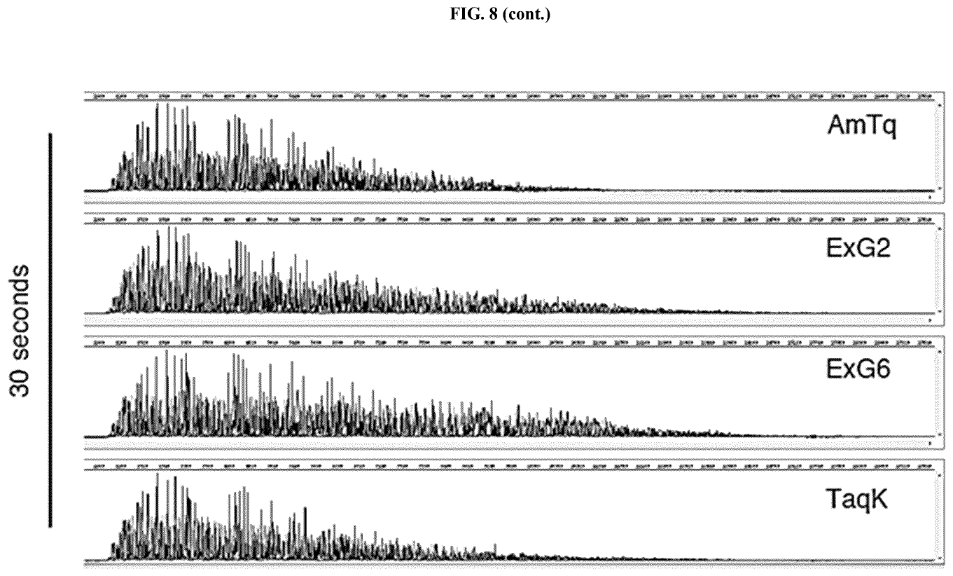

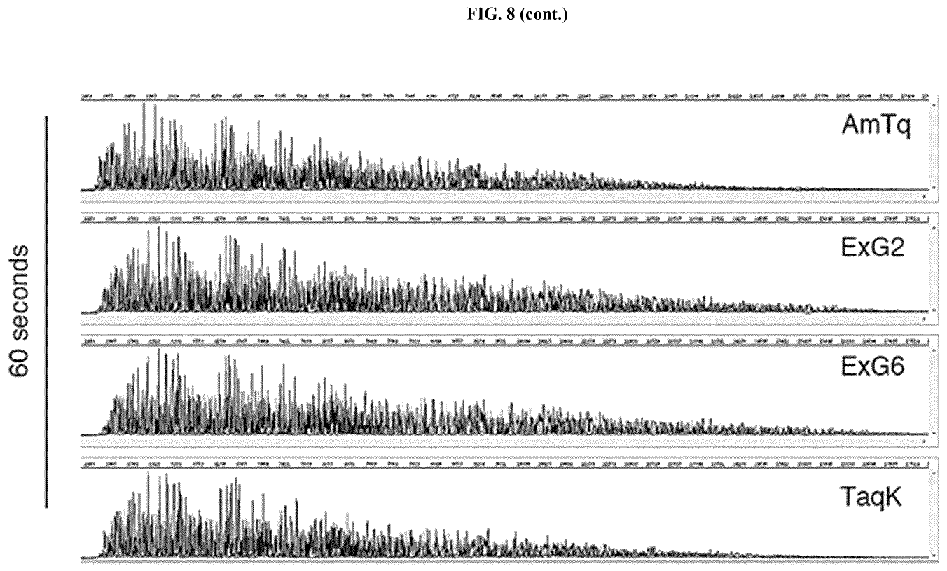

The results of the Sanger sequencing assay are provided in FIG. 8. Elongation times of 10, 30, and 60 seconds show that ExG6 and ExG2 show markedly improved nucleotide incorporation rates compared to AmTq.

### Example 10: Affinity and Catalytic Rate Determination for Taq DNA Polymerases

An affinity and catalytic rate determination was conducted for four Taq polymerases: AM (i.e., AmpliTaq FS), ExG2, ExG6, and TaqK. Their binding kinetics and catalytic/DNA elongation activity were measured using a switchSENSE® DRX² automated analyzer.

An 80mer red DNA lever was hybridized with 500 nm 48mer primer as the ligand. The Taq polymerase being tested was then used to treate the hybridized DNA at 100 μl/min. The association constant was determined, and the complex was treated with dNTPs at 500 μl/min to determine elongation activity. The newly formed nucleotide was then removed with a pH 13 wash.

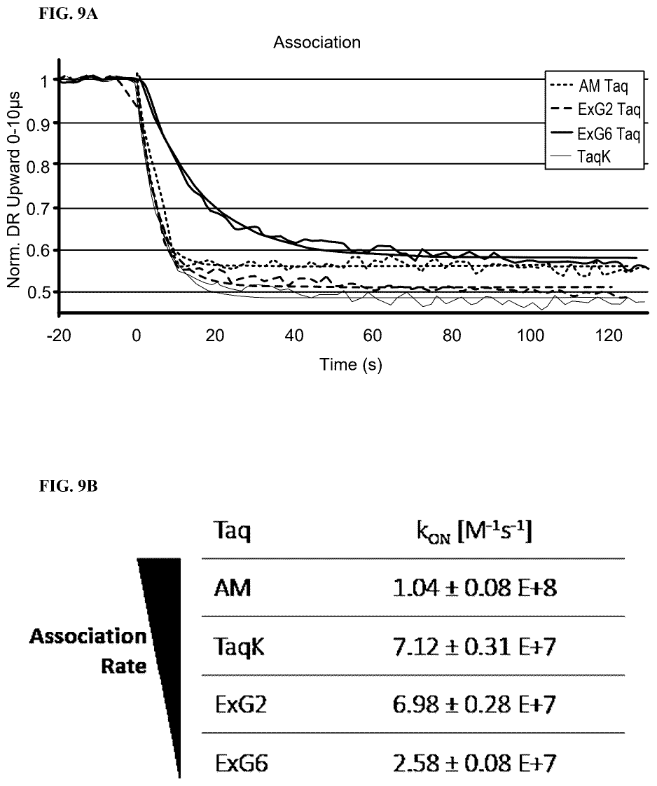

The results of the kinetic and catalytic activity determinations are provided in FIGS. 9A, 9B, 10A, 10B, 11, 12A-12C, and 13.

FIG. 9A-FIG. 9B is a comparison of the kinetic association rates (k_{ON}) for the Taq polymerase variants ExG2, ExG6, and TaqK and the commercial enzyme AmpliTaq (AM AmTq; AM). The k_{ON} ranking for the Taq constructs was AM>TaqK>ExG2>ExG6.

FIG. 10A-FIG. 10B is a comparison of the kinetic disassociation (k_{OFF}) and surface recovery ranking (a_{OFF}) for the Taq polymerase variants ExG2, ExG6, and TaqK and the commercial enzyme AM. The surface recovery ranking for Taq constructs (from weaker to stronger affinity) is AM>ExG6>ExG2>TaqK.

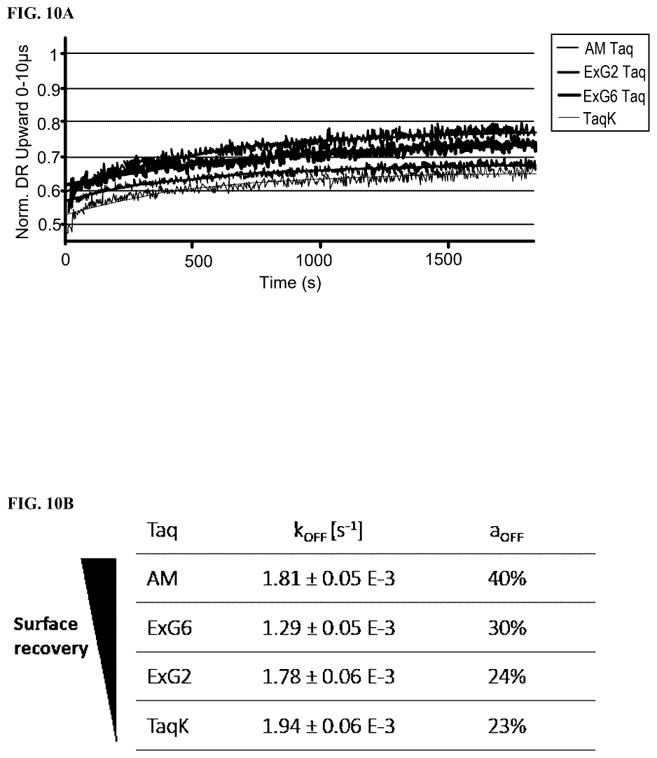

FIG. 11 is a comparison of the kinetic association (k_{OFF}) and disassociation (k_{OFF}) rates for the Taq polymerase variants ExG2, ExG6, and TaqK and the commercial enzyme AM.

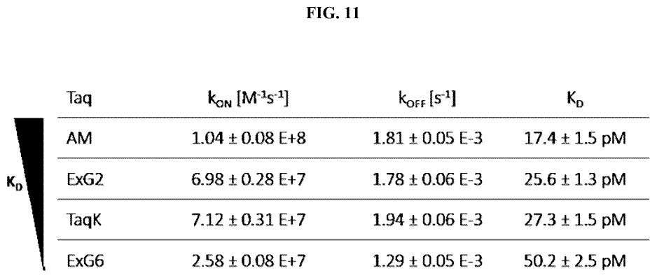

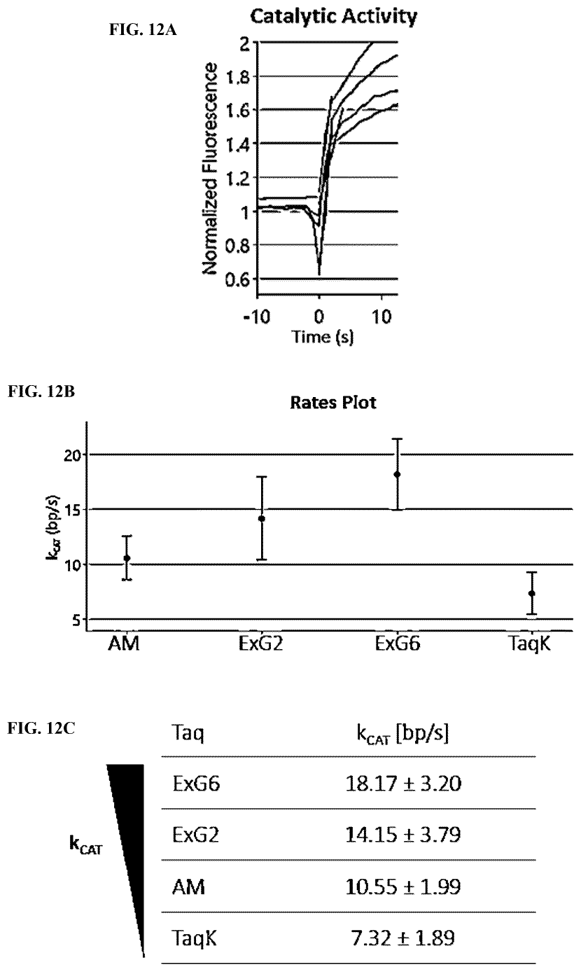

FIG. 12A-FIG. 12C is a comparison of the catalytic activity rates (k_{CAT}) for the Taq polymerase variants ExG2, ExG6, and TaqK and the commercial enzyme AM. The k_{CAT} ranking for the Taq polymerase variants is ExG6>ExG2>AM>TaqK.

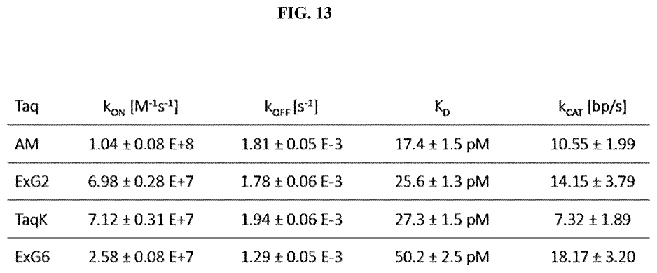

FIG. 13 summarizes the binding kinetics and catalytic activity rates for the Taq polymerase variants ExG2, ExG6, and TaqK and the commercial enzyme AM.

All features of the described compositions and/or kits are applicable to the described methods mutatis mutandis, and vice versa.

All patent filings, scientific journal articles, books, treatises, and other publications and materials discussed or cited in this application are hereby incorporated by reference in their entirety for all purposes.

Where a range of values is provided, it is understood that each intervening value between the upper and lower limits of that range is also specifically disclosed, to the smallest fraction of the unit or value of the lower limit, unless the context dictates otherwise. Any encompassed range between any stated value or intervening value in a stated range and any other stated or intervening value in that stated range is disclosed. The upper and lower limits of those smaller ranges may independently be included or excluded in the range, and each range where either, neither, or both limits are included in the smaller range is also disclosed and encompassed within the technology, subject to any specifically excluded limit, value, or encompassed range in the stated range. Where the stated range includes one or both of the limits, ranges excluding either or both of those included limits are also included.

It is to be understood that the figures and descriptions of the disclosure have been simplified to illustrate elements that are relevant for a clear understanding of the disclosure. It should be appreciated that the figures are presented for illustrative purposes and not as construction drawings. Omitted details and modifications or alternative embodiments are within the purview of persons of ordinary skill in the art.

It can be appreciated that, in certain aspects of the disclosure, a single component may be replaced by multiple components, and multiple components may be replaced by a single component, to provide an element or structure or to perform a given function or functions. Except where such substitution would not be operative to practice certain embodiments, such substitution is considered within the scope of the disclosure.

The examples presented herein are intended to illustrate potential and specific implementations of the invention. It can be appreciated that the examples are intended primarily for purposes of illustration for those skilled in the art. There may be variations to these diagrams or the operations described herein without departing from the spirit of the invention. For instance, in certain cases, method steps or operations may be performed or executed in differing order, or operations may be added, deleted or modified.

Different arrangements of the components depicted in the drawings or described above, as well as components and steps not shown or described are possible. Similarly, some features and sub-combinations are useful and may be employed without reference to other features and sub-combinations. Aspects and embodiments of the invention have been described for illustrative and not restrictive purposes, and alternative embodiments will become apparent to readers of this patent. Accordingly, the present invention is not limited to the embodiments described above or depicted in the drawings, and various embodiments and modifications can be made without departing from the scope of the claims below.

While exemplary embodiments have been described in some detail, by way of example and for clarity of understanding, those of skill in the art will recognize that a variety of modification, adaptations, alternate constructions, equivalents and changes may be employed. Hence, the scope of the present invention should be limited solely by the claims.

1. A Thermus aquaticus (Taq) DNA polymerase, wherein the Taq DNA polymerase comprises an F667Y substitution and at least one substitution selected from the group consisting of E507K, S543N, E742H, and A743H; and wherein the Taq DNA polymerase retains 5′ to 3′ exonuclease activity.

2. The polymerase of claim 1, wherein the Taq DNA polymerase has at least one substitution selected from the group consisting of E742H, A743H, and S543N.

3. The polymerase of claim 1, wherein the Taq DNA polymerase has an E742H substitution and an A743H substitution.

4. The composition of claim 1, wherein the Taq DNA polymerase hasan S543N substitution.

5. The composition of claim 1, wherein the Taq DNA polymerase has improved primer extension elongation as compared to AmpliTaq FS™ (SEQ ID NO: 21).

6. The composition of claim 1, wherein the Taq DNA polymerase has improved Sanger sequencing elongation as compared to AmpliTaq FS™ (SEQ ID NO: 21).

7. The composition of claim 1, further comprising the substitution E507K.

8. The composition of claim 1, further comprising a substitution G46D.

9. The composition of claim 1, further comprising a substitution M747K.

10. The composition of claim 1,
further comprising a histidine purification tag.

11. The composition of claim 10, wherein the histidine purification tag comprises the sequence ASENLYFQGHHHHHH (SEQ ID NO: 35).

12. The composition of claim 1, further comprising deletion of one or more amino acids of wild-type sequence positions 1-11.

13. The composition of claim 12, wherein the deletion is an R2 deletion.

14. The composition of claim 1, wherein the N-terminal sequence comprises a pIVc sequence and an optional linker.

15. The composition of claim 13, wherein the pIVc sequence comprises the sequence GVQSLKRRRCF (SEQ ID NO: 37).

16. The composition of claim 13, wherein the optional linker comprises the sequence GGGVTS (SEQ ID NO: 39).

17. The composition of claim 15, wherein the N-terminal sequence comprises the sequence MGVQSLKRRRCFGGGVTSGMLP (SEQ ID NO: 41).

18. The composition of claim 1, further comprising a pyrophosphatase.

19. The composition of claim 1, wherein the Taq DNA polymerase has increased 5′ to 3′ exonuclease activity as compared to AmpliTaq FS™ (SEQ ID NO: 21).

20. The composition of claim 1, wherein the composition has improved processivity and/or stand displacement activity as compared to AmpliTaq FS™ (SEQ ID NO: 21).

21. The composition of claim 1, wherein the composition can incorporate a dideoxynucleotide triphosphate (ddNTP) at the 3′ end of a primer or a nucleic acid molecule.

22. The composition of claim 1, wherein the composition does not discriminate between incorporation of a deoxynucleotide triphosphate (dNTP) or a dideoxynucleotide triphosphate (ddNTP) at the 3′ end of a primer or a nucleic acid molecule by more than 5-fold.

23. The composition of claim 1, wherein the Taq DNA polymerase is a thermostable DNA polymerase.

24-51. (canceled)
